(19) United States
(12) Patent Application Publication
(10) Pub. No.: US 2022/0315588 A1
(43) Pub. Date: Oct. 6, 2022

(54) ALTERNATIVE PROCESS FOR THE PREPARATION OF 4-PHENYL-5-ALKOXYCARBONYL-2-THIAZOL-2-YL-1,4-DIHYDROPYRIMIDIN-6-YL]METHYL]-3-OXO-5,6,8,8A-TETRAHYDRO-1H-IMIDAZO[1,5-A]PYRAZIN-2-YL]-CARBOXYLIC ACID

(71) Applicant: Hoffmann-La Roche Inc., Little Falls, NJ (US)

(72) Inventors: Daniel Vincent FISHLOCK, Basel (CH); Jianshu LIU, Changzhou (CN); Paul SPURR, Riehen (CH); Georg WUITSCHIK, Basel (CH); Zhixiang XU, Changzhou (CN); Fugui ZHANG, Changzhou (CN)

(73) Assignee: Hoffmann-La Roche Inc., Little Falls, NJ (US)

(21) Appl. No.: 17/616,930

(22) PCT Filed: Jun. 4, 2020
(86) PCT No.: PCT/EP2020/065424

§ 371 Date: Dec. 6, 2021

(30) Foreign Application Priority Data
Jun. 6, 2019 (CN) PCT/CN2019/090358

(51) Int. Cl. C07D 487/04

(52) U.S. Cl. CPC C07D 487/04

(57) ABSTRACT

The present invention relates to an alternative process for synthesizing a compound of formula (I), R¹ is phenyl, which is unsubstituted or substituted with one, two or three substituents independently selected from halogen and C₁₋₆alkyl; R² is C₁₋₆alkyl; R³ is —CₓH₂—; x is 1, 2, 3, 4, 5, 6 or 7; or pharmaceutically acceptable salt or diastereomer thereof, which is useful for prophylaxis and treatment of a viral disease in a patient relating to hepatitis B infection or a disease caused by hepatitis B infection.

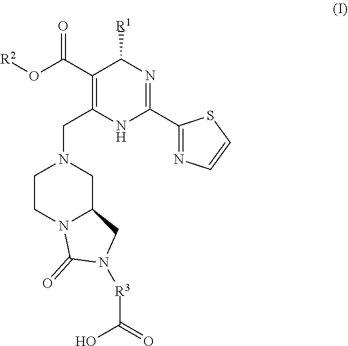

The present invention relates to an alternative process for the preparation of a compound of formula (Ia),

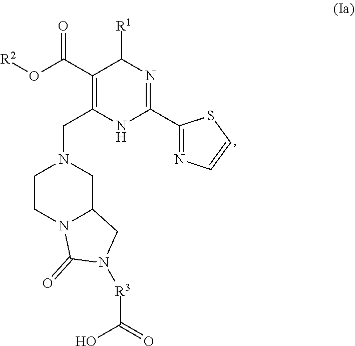

particularly a compound of formula (I),

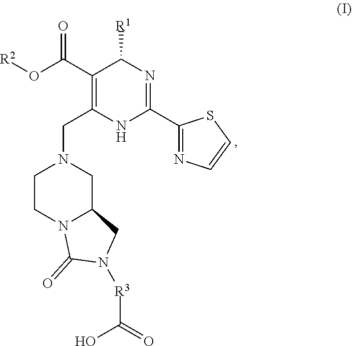

wherein

R¹ is phenyl, which is unsubstituted or substituted with one, two or three substituents independently selected from halogen and C₁₋₆alkyl;

R² is C₁₋₆alkyl;

R³ is —CₓH₂ₓ—;

x is 1, 2, 3, 4, 5, 6 or 7;

or pharmaceutically acceptable salt or diastereomer thereof, which is useful for prophylaxis and treatment of a viral disease in a patient relating to hepatitis B infection or a disease caused by hepatitis B infection.

### BACKGROUND OF THE INVENTION

An approach for synthesizing compounds of formula (I) was disclosed in patent WO 2015/132276. However, the synthetic approach is not suitable for a commercial process due to a number reasons which among others include (i) an overall low yield, (ii) expensive starting materials, (iii) cumbersome stereochemical separation and purification of chiral intermediates and the final product, and (iv) lack of robustness of the Swern oxidation step.

A more efficient synthetic approach which could also be applied on a technical scale and which allows for higher product yield and stereochemical purity was disclosed in WO 2017/140750.

The present invention now discloses a further modified synthetic approach for preparing a compound of formula (Ia) and in particular a compound of formula (I) suitable on an industrial scale which has a further reduced number of steps of the overall process, substantially reduces waste generation and is therefore more favorably in terms of overall costs compared to the processes previously described.

A first aspect of the present invention relates to a novel process for the preparation of a compound of the formula (X):

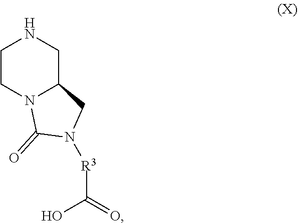

wherein R³ is —CₓH₂ₓ—; x is 1, 2, 3, 4, 5, 6 or 7; or pharmaceutically acceptable salt, enantiomer or diastereomer thereof.

A second aspect of the present invention relates to a novel process for the preparation of a compound of formula (XVIII)

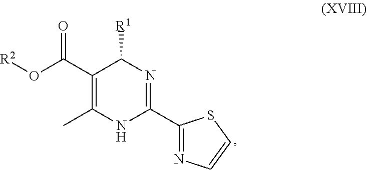

wherein R¹ is phenyl, which is unsubstituted or substituted with one, two or three substituents independently selected from halogen and C₁₋₆alkyl; R² is C₁₋₆alkyl; or pharmaceutically acceptable salt, enantiomer or diastereomer thereof.

Compound of the formulae (X) and (XIX) are key intermediates in the synthesis and manufacture of pharmaceutically active compound of formula (I) as described herein.

A third aspect of the present invention relates to a novel process for the preparation of a compound of formula a compound of formula (Ia),

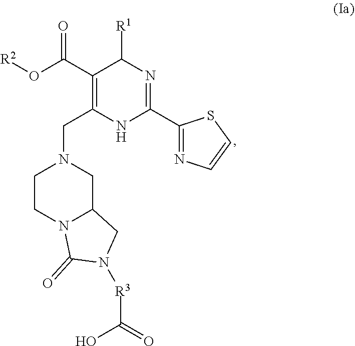

and in particular a compound of formula (I),

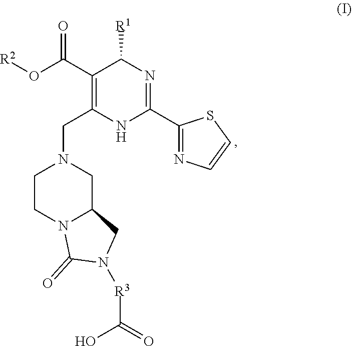

wherein

R¹ is phenyl, which is unsubstituted or substituted with one, two or three substituents independently selected from halogen and C₁₋₆alkyl;

R² is C₁₋₆alkyl;

R³ is —CₓH₂ₓ—;

x is 1, 2, 3, 4, 5, 6 or 7;

or pharmaceutically acceptable salt or diastereomer thereof.

### DETAILED DESCRIPTION OF THE INVENTION

### Definitions

As used herein, the term “C₁₋₆alkyl” signifies a saturated, linear- or branched chain alkyl group containing 1 to 6, particularly 1 to 5 carbon atoms, for example methyl, ethyl, n-propyl, isopropyl, n-butyl, isobutyl, tert-butyl and the like. Particularly, “C₁₋₆alkyl” group is methyl or ethyl.

The term “halogen” signifies fluorine, chlorine, bromine or iodine, particularly fluorine or chlorine.

The term “diastereomer” denotes a stereoisomer with two or more centers of chirality and whose molecules are not mirror images of one another.

The term “pharmaceutically acceptable salt” refers to conventional acid-addition salts or base-addition salts that retain the biological effectiveness and properties of the compounds of formula I and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435; or in Ansel, H., et al., In: Pharmaceutical Dosage Forms and Drug Delivery Systems, 6th ed. (1995), pp. 196 and 1456-1457.

### Abbreviation

ACN Acetonitrile

API active pharmaceutical ingredient

Boc tert-Butoxycarbonyl

(R)-BNP acid (R)-(−)-1,1′-Binaphthyl-2,2′-diyl hydrogen phosphate

CPME Cyclopentyl methyl ether

DBU 1,8-Diazabicyclo[5.4.0]undec-7-ene

DCM dichloromethane

DIPEA N,N-Diisopropylethylamine

eq Equivalent

GABA γ-aminobutyric acid

IPA Isopropanol

IPAc Isopropyl acetate

EtOAc or EA ethyl acetate

MEK 2-Butanone

2-MeTHF 2-Methyltetrahydrofuran

MIBK Methyl isobutyl ketone

MSA Methanesulfonic acid

MTBE Methyl tert-butyl ether

NBS N-bromosuccinimide

NMM N-methylmorpholine

TEA Triethylamine

TFA Trifluoroacetic acid

THF tetrahydrofuran

TMP 2,2,6,6-Tetramethylpiperidine

v/v Volume ratio

V65 2,2′-Azobis-(2,4-dimethylvaleronitrile)

wt % Weight percentage

The present invention provides a process for preparing the compounds of formula (X) as outlined in the Scheme 1 and compounds of formulae (XVIII) and (I) as outlined in the Scheme 2.

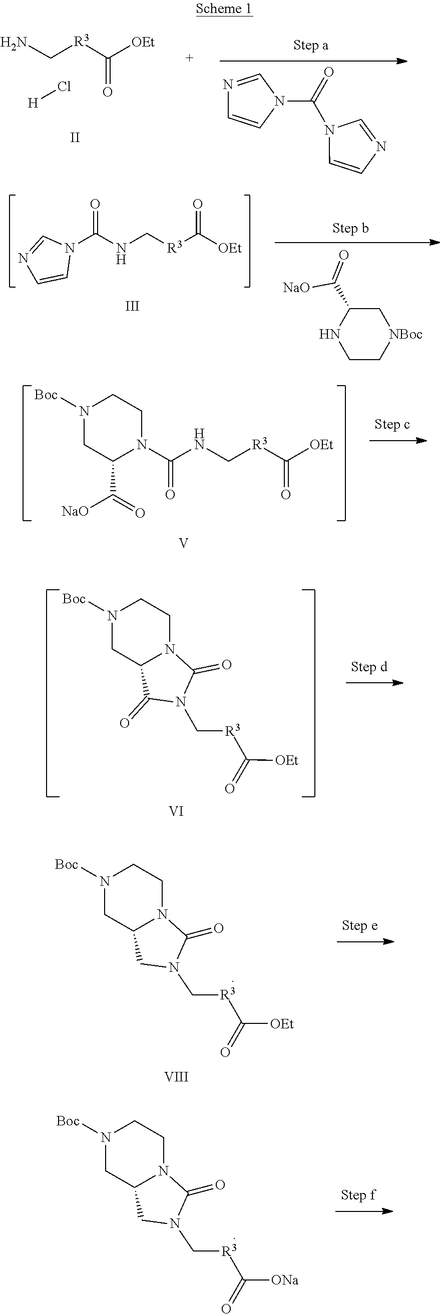

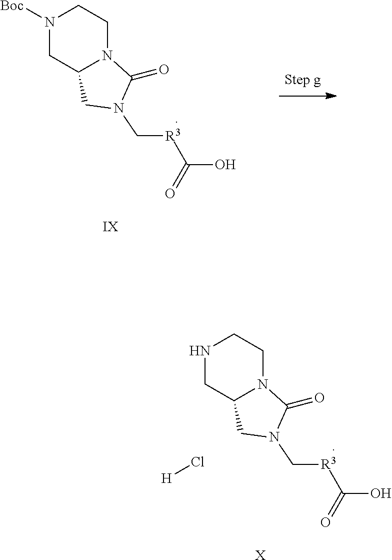

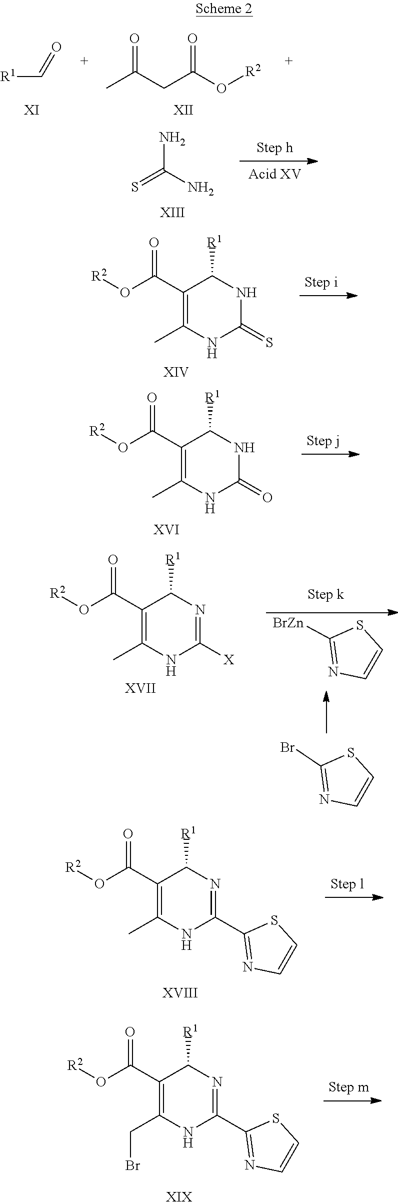

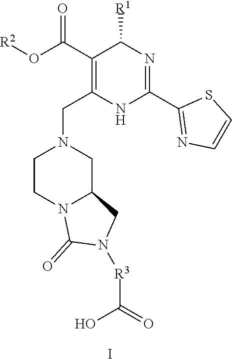

wherein R¹ is phenyl, which is unsubstituted or substituted with one, two or three substituents independently selected from halogen and C₁₋₆alkyl; R² is C₁₋₆alkyl; R³ is —CₓH₂ₓ—; x is 1, 2, 3, 4, 5, 6 or 7; Acid (XV) is (R)-3,3′-Bis(2,4,6-triisopropylphenyl)-1,1′-binaphthyl-2,2′-diyl hydrogenphosphate, (S)-3,3′-Bis(2,4,6-triisopropylphenyl)-1,1′-binaphthyl-2,2′-diyl hydrogenphosphate, (R)-(−)-3,3′-Bis(triphenylsilyl)-1,1′-binaphthyl-2,2′-diyl hydrogenphosphate, (R)-(−)-VAPOL hydrogenphosphate, (+)-CSA, or (S)-(+)-1,1′-Binaphthyl-2,2′-diyl hydrogen phosphate, (R)-(−)-1,1′-Binaphthyl-2,2′-diyl hydrogen phosphate. Preferably, the acid of formula (XV) which functions as catalyst in step h) is (R)-(−)-3,3′-Bis(triphenylsilyl)-1,1-binaphthyl-2,2′-diyl hydrogenphosphate.

The synthesis comprises one or more of the following steps:

step a) the formation of compound (III),

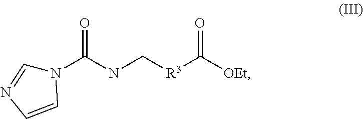

wherein R³ is —CₓH₂ₓ—; x is 1, 2, 3, 4, 5, 6 or 7;

step b) the formation of urea (V)

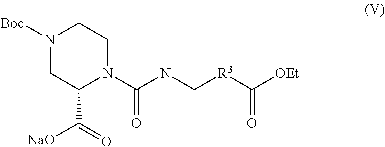

via the addition reaction of compound (III) and compound (IV)

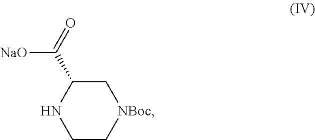

wherein R³ is —CₓH₂ₓ—; x is 1, 2, 3, 4, 5, 6 or 7;

step c) the formation of the hydantoin of formula (VI) via the cyclization reaction of urea (V),

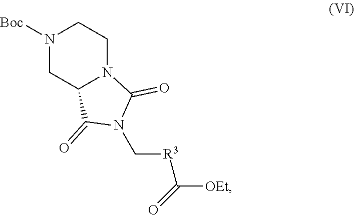

wherein R³ is —CₓH₂ₓ—; x is 1, 2, 3, 4, 5, 6 or 7;

step d) the formation of the urea of formula (VIII) via selective reduction of the compound of formula (VI),

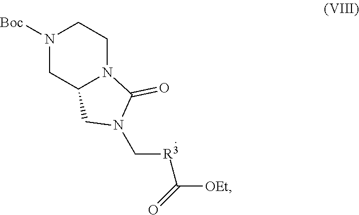

wherein R³ is —CₓH₂ₓ—; x is 1, 2, 3, 4, 5, 6 or 7; R is C₁₋₆alkyl;

steps e) and f) the formation of the compound of formula (IX) via hydrolysis of the compound of formula (VIII),

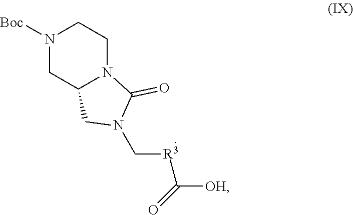

wherein R³ is —CₓH₂ₓ—; x is 1, 2, 3, 4, 5, 6 or 7; R is C₁₋₆alkyl;

step g) the formation of compound of formula (X) by de-protection of the compound of formula (IX),

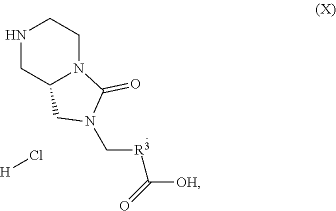

wherein R³ is —CₓH₂ₓ—; x is 1, 2, 3, 4, 5, 6 or 7;

step h) the formation of compound of formula (XIV) via the reaction of compounds (XI), (XII) and (XIII) in the presence of acid (XV),

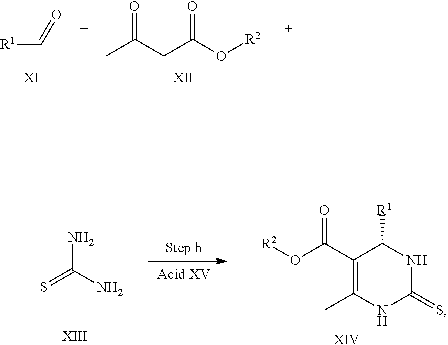

wherein R¹ is phenyl, which is unsubstituted or substituted with one, two or three substituents independently selected from halogen and C₁₋₆alkyl; R² is C₁₋₆alkyl;

step i) the formation of compound of formula (XVI),

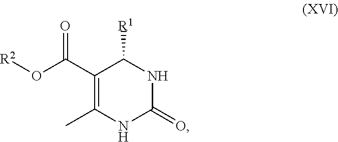

wherein R¹ is phenyl, which is unsubstituted or substituted with one, two or three substituents independently selected from halogen and C₁₋₆alkyl; R² is C₁₋₆alkyl;

step j) the formation of compound of formula (XVII),

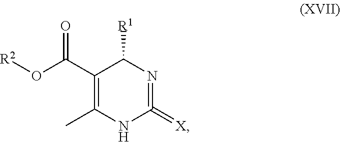

wherein R¹ is phenyl, which is unsubstituted or substituted with one, two or three substituents independently selected from halogen and C₁₋₆alkyl; R² is C₁₋₆alkyl; X is halogen, preferably chlorine;

step k) the formation of compound of formula (XVIII),

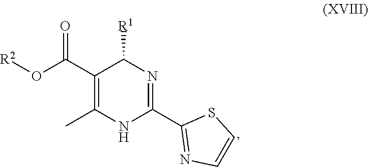

wherein R¹ is phenyl, which is unsubstituted or substituted with one, two or three substituents independently selected from halogen and C₁₋₆alkyl; R² is C₁₋₆alkyl;

step l) the formation of compound of formula (XIX) via the bromination reaction of compound of formula (XVIII),

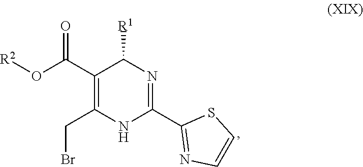

wherein R¹ is phenyl, which is unsubstituted or substituted with one, two or three substituents independently selected from halogen and C₁₋₆alkyl; R² is C₁₋₆alkyl;

step m) the formation of compound of formula (I) via the substitution reaction of compound of formula (XIX) with compound of formula (X),

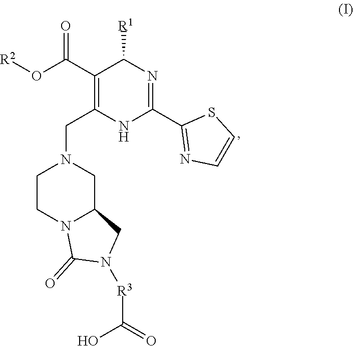

wherein R¹ is phenyl, which is unsubstituted or substituted with one, two or three substituents independently selected from halogen and C₁₋₆alkyl; R² is C₁₋₆alkyl; R³ is —CₓH₂ₓ—; x is 1, 2, 3, 4, 5, 6 or 7.

A detailed description of present invention of process steps is as following:

Step a) the formation of compound (III).

Compound (III) is formed in the presence of a suitable base in a suitable solvent from compound (II) and a suitable reagent, preferably 1,1′-carbonyldiimidazole (CDI). The conversion as a rule is performed under a cooling condition.

The suitable solvent is selected from 2-MeTHF, THF, IPAc, EA, DCM, DMF, toluene and anisole, particularly the suitable solvent is anisole.

The suitable base is selected from Na₂CO₃, NaOtPent, K₂CO₃, Na₃PO₄, K₃PO₄ and triethylamine (TEA). Preferably, the suitable base is TEA. The rate of the reaction is controlled at a temperature between −20° C. and 40° C., particularly between 0° C. and 5° C.

The suitable reagent is selected from CDI, phosgene, diphosgene, disuccinimidyl carbonate, and triphosgene, preferably the reagent is CDI. The amount of CDI is from 1.0 to 2.0 eq. of compound of formula (II), particularly 1.1 to 1.5 eq.

WO 2017/140750 discloses an alternative synthetic path for making compound X which uses a phosgene reagent in the formation of an isocyanate intermediate. The phosgene reagent is selected from phosgene, diphosgene and triphosgene. It is well known in the art that all those phosgene reagents are highly toxic. The synthetic process according to the present invention avoids any phosgene reagent and instead uses for instance CDI in step a).

Step b) the formation of urea (V) via the addition reaction of compounds (III) and (IV).

The urea (V) is synthesized in a suitable organic solvent. The conversion as a rule is performed under a mild heating condition.

The condensation reaction is conducted in a suitable organic solvent, which is selected from 2-MeTHF, THF, IPAc, EA, DMF, anisole, toluene and DCM. Particularly the solvent is anisole

The reaction is performed at temperature between 0° C. and 80° C., particularly between 0° C. and 60° C., more particularly between 30° C. and 50° C.

In the present synthesis,

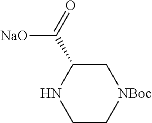

is used in step b) instead of

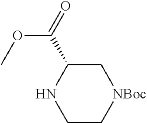 [SMILES: COC(=O)[C@@H]1CN(C(=O)OC(C)(C)C)CCN1]

as in the previously described synthesis (WO 2017/140750). The sodium compound is substantially cheaper than the methoxy compound used in the previously described synthesis. Because of the presence of the free NH, it is more cumbersome to make the ester from the free acid (requires several steps). Thus, the sodium salt is substantially lot cheaper.

Step c) the formation of the hydantoin of formula (VI) via the cyclization reaction of urea (V).

The compound of formula (VI) is synthesized via the cyclization of urea (V) in the presence of a suitable acid in a suitable organic solvent. The conversion as a rule is performed under a cooling condition.

The suitable solvent is selected from 2-MeTHF, IPAc, EA, toluene, DCM, anisole, and DMF. Preferably the solvent is anisole

The suitable acidic dehydrating agent is selected from boron trifluoride etherate, phosphoric acid, sulphuric acid, chlorosulphonic acid, trifluoroacetic acid, HBr, HCl, AlCl₃, TiCl₄, SnCl₄, ZrCl₄, TMSOTf, pivaloyl chloride, isobutyl chloroformate and oxalyl chloride. Preferably, the acidic dehydrating agent is oxalyl chloride. The reaction is performed at temperatures between −20° C. and 20° C., particularly between −5° C. and 5° C.

Step d) the formation of the urea of formula (VIII) via selective reduction of the compound of formula (VI).

The compound of formula (VIII) is synthesized in the presence of a suitable catalytic Lewis acid and a suitable reducing agent in a suitable solvent. The conversion is performed under a cooling condition.

The suitable solvent is selected from THF, 2-MeTHF and cyclopentyl methyl ether, particularly the solvent is THF or 2-MeTHF or anisole.

The suitable reducing agent is selected from lithium aluminum hydride, sodium dihydro-bis-(2-methoxyethoxy)aluminate, borane dimethylsulfide, phenylsilane, borane, borane dimethylsulphide complex and borane tetrahydrofuran complex, particularly the reductive reagent is borane tetrahydrofuran complex. The amount of borane tetrahydrofuran complex is 1.6-5.0 eq. of the compound of formula (VI), particularly 1.6-2.0 eq.

The catalytic Lewis acid is selected from InCl₃, YCl₃, ZnCl₂, ZnCl₂, TMSCl, TiCl₄, ZrCl₄, AlCl₃, BF₃.THF, and BF₃.Et₂O, particularly the Lewis acid is BF₃.Et₂O. The amount of BF₃.Et₂O is 0.05-1.1eq. of the compound of formula (VI), particularly 0.2 eq.

The reaction is performed at a reaction temperature between −40 and 40° C., particularly between 10° C. and 15° C.

Usually 4-5 eq. of borane tetrahydrofuran complex can give 100% conversion but suffer from poor selectivity of reduction over other carbonyl groups. With catalytic amounts of BF₃.Et₂O, not only the selectivity is improved but also the amount of borane tetrahydrofuran complex is decreased from 4-5 eq. to 1.6-2.0 eq.

Steps e) and f) the formation of the compound of formula (IX) via hydrolysis of the compound of formula (VIII).

The compound of formula (IX) is synthesized in the presence of a suitable base in a suitable solvent followed by a work-up procedure.

The suitable solvent is selected from THF, MeTHF, TBME, toluene, anisole, isopropanol, methanol and ethanol and their mixtures with water. Particularly the solvent is a mixture of water andanisole.

The suitable base for hydrolysis is selected from LiOH, LiOOH, NaOTMS, KOTMS, KOtBu, NaOH and KOH. Particularly the base is aq. NaOH.

The reaction is performed at temperature between 0° C. and 70° C., particularly between 40° C. and 60° C.

The compound of formula (IX) is isolated through a work-up procedure comprising of phase separation, acidification and isolation of the resulting free acid.

In one embodiment of the present invention, steps a) to f) will be carried out in a single reaction vessel as a so-called one-pot synthesis. This circumvents several purification procedures of the intermediates formed in relation to steps a) to f) and thereby minimizing chemical waste, saving time and simplifying other aspects of the chemical process like reducing energy consumption and use of equipment.

Step g) the formation of compound of formula (X) by deprotection of the compound of formula (IX).

Compound of formula (X) is synthesized in the presence of a suitable acid in a suitable solvent.

The suitable solvent is selected from DCM, toluene, dioxane, EtOAc, IPAc, IPA, 1-propanol, acetone, MIBK and mixed solvent of MIBK and acetone. Particularly the solvent is MIBK.

The suitable acid is selected from TFA, phosphoric acid, MSA, sulphuric acid, HBr and HCl. Particularly the acid is TFA or HCl, and more particularly the acid is HCl.

The addition rate of the acid is controlled while the reaction temperature is maintained between 0° C. and 60° C., particularly between 20° C. and 30° C. while the gas release can be controlled.

The amount of acid is 3-10 eq. of the compound of formula (IX), particularly 3-4 eq.

After an appropriate amount of time, usually 0.5-2 hours, the reaction is completed with monitoring by HPLC. The compound of formula (X) is isolated as a solid from the reaction mixture. The compound of formula (X) precipitates in the reaction mixture and is separated by filtration followed by one or more washing steps using the solvent in which the reaction had been carried out.

One aspect of the present invention relates to a synthetic process for making the compound of formula (X) comprising at least one of the steps a) to g).

Step h) the formation of compound of formula (XIV) via the reaction of compounds (XI), (XII) and (XIII) in the presence of acid (XV).

Compound of formula (XIV) is synthesized in the presence of a suitable catalyst in a suitable solvent. The conversion as a rule is performed under Dean-Stark water removal conditions (reduced pressure).

The suitable solvent is selected from methanol, ethanol, IPA, tert-BuOH, 2,2,2-trifluroethanol, benzene, xylene, anisole, chlorobenzene and toluene, particularly the solvent is toluene.

The suitable organic acid catalyst used in the enantioselective Biginelli reaction is selected from (S)-(+)-3,3′-Bis(triphenylsilyl)-1,1′-binaphthyl-2,2′-diyl hydrogen-phosphate, (R)-(−)-3,3′-Bis(triphenylsilyl)-1,1′-binaphthyl-2,2′-diyl hydrogen-phosphate, D-(+)-DTTA, L-DTTA, L-Tartaric acid, D-DBTA, (+)-CSA, (S)-(+)-1,1′-Binaphthyl-2,2′-diyl hydrogen phosphate and (R)-(−)-1,I-Binaphthyl-2,2′-diyl hydrogen phosphate, (R)-3,3′-Bis(2,4,6-triisopropylphenyl)-1,1′-binaphthyl-2,2′-diyl hydrogenphosphate, (S)-3,3′-Bis(2,4,6-triisopropylphenyl)-1,1′-binaphthyl-2,2′-diyl hydrogenphosphate, (R)-(−)-VAPOL hydrogenphosphate particularly the organic acid is (R)-(−)-3,3′-Bis(triphenylsilyl)-1,1′-binaphthyl-2,2′-diyl hydrogen-phosphate.

WO 2017/140750 discloses an alternative synthetic path for making compound (XIX) wherein in the formation and recrystallization of the enantiomeric salt of the compound of formula (XVI) preferably either (S)-(+)-1,1′-Binaphthyl-2,2′-diyl hydrogen phosphate or (R)-(−)-1,1′-Binaphthyl-2,2′-diyl hydrogen phosphate is used. In one embodiment of the present invention, either (S)-(+)-3,3′-Bis(triphenylsilyl)-1,1′-binaphthyl-2,2′-diyl hydrogen-phosphate or (R)-(−)-3,3′-Bis(triphenylsilyl)-1,1′-binaphthyl-2,2′-diyl hydrogen-phosphate, preferably (R)-(−)-3,3′-Bis(triphenylsilyl)-1,1′-binaphthyl-2,2′-diyl hydrogen-phosphate is used in the step h) wherein the compound of formula (XIV) is formed enantiospecifically. In contrast to the teaching of WO 2017/140750 wherein equimolar amounts of either (S)-(+)-3,3′-Bis(triphenylsilyl)-1,1′-binaphthyl-2,2′-diyl hydrogen-phosphate or (R)-(−)-3,3′-Bis(triphenylsilyl)-1,1′-binaphthyl-2,2′-diyl hydrogen-phosphate are necessary, the amount of the corresponding 1,1′-Binaphthyl-2,2′-diyl hydrogen phosphate needed in the process step h) according to the present invention is just 0.01 equimolar. Therefore, a substantial reduction of process waste and costs over the processes previously described in the art is possible with the synthetic path according to the present invention.

Step i) the formation of compound of formula (XVI).

Compound of formula (XVI) is synthesized in the presence of a suitable catalyst at a suitable pH using a suitable reagent in a suitable solvent.

The suitable solvent is selected from mixtures of water with two of either methanol, ethanol, 2,2,2-trifluroethanol, toluene, ACN, DMF, EtOAc or dimethyl carbonate, particularly the solvent is a mixture of water, ethanol and ACN.

The suitable reagent used in the reaction is selected from sodium carbonate, potassium carbonate, sodium hydrogencarbonate, potassium hydrogencarbonate, formic acid, acetic acid, particularly the catalyst is sodium hydrogencarbonate.

The suitable pH for this reaction is between 5 and 12, particularly the pH is between 7 and 10.

The suitable reagent used in the reaction is selected from mCPBA, tBuOOH, urea hydrogen peroxide complex, dibenzoyl peroxide, oxone, and an aqueous solution of hydrogen peroxide, particularly the reagent is an aqueous solution of hydrogen peroxide.

Step j) the formation of compound of formula (XVII).

Compound of formula (XVII) is synthesized using a suitable reagent in a suitable solvent.

The suitable solvent is selected from toluene, xylenes, chlorobenzene, heptane, ACN, dichloromethane, particularly the solvent is toluene.

The suitable reagent is selected from oxalyl chloride, PCl₅, POCl₃, SOCl₂, and MsCl, particularly the reagent is POCl₃.

Step k) the formation of compound of formula (XVIII).

Compound of formula (XVIII) is synthesized using a suitable catalyst and a suitable reagent in a suitable solvent and isolated as a suitable salt, preferably as the HBr salt.

The suitable catalyst is selected from complexes of either Xantphos or dppf with Palladium(II)-salts, particularly the catalyst is XantphosPdCl₂.

The suitable reagent is selected from bromo(thiazol-2-yl)magnesium, thiazol-2-ylboronic acid and bromo(thiazol-2-yl)zinc, particularly the reagent is bromo(thiazol-2-yl)zinc.

The suitable solvent is selected from toluene, xylenes, chlorobenzene, THF, 2-Methyltetrahydrofurane, ACN, dichloromethane, particularly the solvent is toluene.

Step l) the formation of compound of formula (XIX) via the bromination reaction of compound of formula (XVIII).

Compound of formula (XVIII) is synthesized in the presence of a suitable bromination reagent with or without a suitable additive in a suitable organic solvent. The conversion as a rule is performed under a heating condition.

The suitable bromination reagent is selected from NBS, bromine, pyridine tribromide and 1,3-dibromo-5,5-dimethylhydantion, particularly the bromination reagent is NBS. The bromination reaction is performed at the temperature between 0° C. and 80° C., particularly between 35° C. and 40° C.

The reaction is usually performed in an organic solvent selected from carbon tetrachloride, 1,2-Dichloroethane, ACN, acetic acid, fluorobenzene, chlorobenzene and DCM, particularly the organic solvent is DCM.

Another aspect of the present invention relates to a synthetic process for making the compound of formula (XIX) comprising at least one of the steps h) to l).

WO 2017/140750 discloses an alternative synthetic path for making compound (XIX). However, the synthetic process according to the present invention is estimated to provide for (i) >50% waste reduction, (ii) >20% lower costs and (iii) a substantially shortened process of ≥3 steps shorter over the process disclosed in WO 2017/140750.

Step m) the formation of compound of formula (I) via the substitution reaction of compound of formula (XIX) with compound of formula (X).

Compound of formula (I) is synthesized in the presence of a suitable base in a suitable organic solvent.

The suitable base is selected from TMP, DIPEA, TEA, tripropylamine, N,N-dicyclohexylmethylamine, DBU, NMM, 2,6-lutidine, 1-methylimidazole, 1,2-dimethylimidazole, tetra methylpiperidine-4-ol, Na₂CO₃, K₂CO₃, NaHCO₃ and tris(2-hydroxylethyl)amine; particularly the base is TMP or tris(2-hydroxylethyl)amine; and more particularly the base is tris(2-hydroxylethyl)amine.

The suitable pKa and nucleophilicity of the base are directly related to the yield and impurities formation in this step. Both TMP and tris(2-hydroxylethyl)amine could result in good yield with high selectivity, but hydrazine related impurities might be introduced to the final API when using TMP as the base.

The suitable organic solvent is selected from THF, IPAc EtOAc, MTBE, fluorobenzene, chlorobenzene and DCM, particularly the organic solvent is DCM.

The substitution reaction as a rule is performed at the temperature between 0° C. and 40° C., particularly at temperature between 10° C. and 25° C.

An efficient purification procedure through an acid-base work-up and recrystallization is needed to ensure the purity of API.

The purification procedure of compound of formula (I) includes: 1) acid-base work-up with a suitable acid and a suitable base in a suitable solvent; and 2) recrystallization which is performed with or without suitable seeding in a suitable organic solvent.

The acid used in the acid-base work-up for purification of compound of formula (I) is selected from HCl, HBr, H₂SO₄, H₃PO₄, MSA, toluene sulfonic acid and camphor sulfonic acid, particularly the acid is H₃PO₄. The concentration of aqueous H₃PO₄ is selected from 15 wt % to 60 wt %; particularly the concentration of aqueous H₃PO₄ is from 35 wt % to 40 wt %. The amount of H₃PO₄ is essential and carefully designed to get the maximum recovery of API and minimum impurities.

The base used in the acid-base work-up for purification of compound of formula (I) is selected from NaOH, KOH, K₂CO₃ and Na₂CO₃, particularly the base is NaOH.

The suitable organic solvent used for extracting impurities in the acid-base work-up for purification of compound of formula (I) is selected from MTBE, EA, IPAc, butyl acetate, toluene and DCM; particularly, the organic solvent is EA or DCM; and more particularly the solvent is DCM.

The suitable solvent for recrystallization of compound of formula (I) is selected from IPA, ethanol, EtOAc, IPAc, butyl acetate, toluene, MIBK, mixed solvent of acetone and water, mixed solvent of IPA and water, and mixed solvent of ethanol and water; particularly the solvent is mixed solvent of ethanol and water. Seeding amount is 0.1-5 wt % of compound of formula (I), particularly the seeding amount is 1 wt %.

### EXAMPLES

### Example 1

### Preparation of C15050794-G (Example 1)

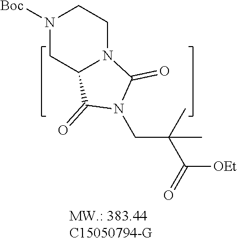

The title compound was prepared according to following scheme:

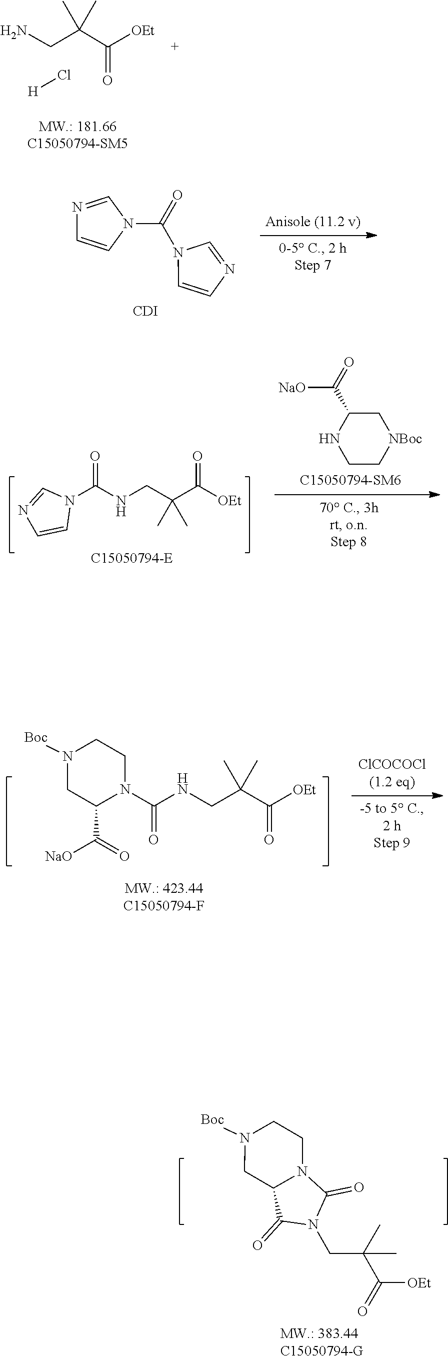

Production of C15050794-G was carried out in two batches. For C15050794-G17601, 1243.4 kg of C15050794-G anisole solution was obtained from 118.35 kg of C15050794-SM6 and 90.0 kg C15050794-SM5 with 92.8% purity, 12.6% assay, 96.6% e.e. in 87% yield. For C15050794-G17602, 1214.6 kg anisole solution of C15050794-G was obtained from 117.35 kg of C15050794-SM6 and 88.9 kg C15050794-SM5 with 93.3% purity, 12.2% assay, 97.5% e.e. in 83% yield. The details are summarized in table below.

### Raw Materials for Preparation of C15050794-G17601

| | | | Quantity | Rel Wt/Vol | | |
|---|---|---|---|---|---|---|
| Batch No. | Material | MW | (kg) | (1X = 118 kg) | Spec | Eq. |
| C13022716-K17401 | C15050794-SM6 | 252.24 | 118 | 1.00 | Purity ≥ 98.0% | 1.00 |
| | (S)-4-(tert- | | | | e.e ≥ 98.0% | |
| | butoxycarbonyl) | | | | | |
| | Piperazine-2- | | | | | |
| | carboxylate | | | | | |
| 17081873 | C15050794-SM5 | 181.66 | 90.0 | 0.76 | Assay ≥ 78% | 1.06 |
| | Ethyl-3-amino-2,2- | | | | | |
| | di-methylpropanoatehydrochloride | | | | | |
| 17032708 | CDI | N/A | 93.3 | 0.79 | Assay ≥ 98% | N/A |
| 15070656 | Oxalyl chloride | N/A | 109 | 0.92 | Purity ≥ 98.0% | N/A |
| 16031116 | Methoxybenzene | N/A | 962 | 8.2 | Purity ≥ 99% | N/A |
| | (anisole) | | | | | |
| 17051771 | Et₃N | N/A | 48.2 | 0.4 | Purity ≥ 99.0% | N/A |
| | | | | | KF ≤ 0.2% | |
| 17081716/17012242 | KHCO₃ | N/A | 249.15 | 2.1 | Assay ≥ 99.0% | N/A |
| 170721/170803/170912 | 25% NaCl aq. | N/A | 2002 | 2002 | N/A | N/A |
| N/A | THF | N/A | 893.26 | 7.57 | Purity ≥ 99.8% | N/A |
| | | | | | KF ≤ 0.05% | |
| 170915/PW-21074 | Process water | N/A | 3182 | 26.97 | pH 6.5-8.5 | N/A |
| 170912/PW-21073 | | | | | | |

### Raw Materials for Preparation of C15050794-G17602

| | | | Quantity | Rel Wt/Vol | | |
|---|---|---|---|---|---|---|
| Batch No. | Material | MW | (kg) | (1X = 118 kg) | Spec | Eq. |
| 17082963/17082965/ | C15050794-SM6 | 252.24 | 117 | 0.99 | Purity ≥ 98.0% | 1.00 |
| 17082964 | (S)-4-(tert- | | | | e.e ≥ 98.0% | |
| | butoxycarbonyl)Piperazine- | | | | | |
| | 2-carboxylate | | | | | |
| 17081873 | C15050794-SM5 | 181.66 | 88.9 | 0.75 | Assay ≥ 78% | 1.06 |
| | Ethyl-3-amino-2,2-di- | | | | | |
| | methylpropanoatehydrochloride | | | | | |
| 17032708 | CDI | N/A | 92.3 | 0.78 | Assay ≥ 98% | N/A |
| 16062306/16072761 | Oxalyl chloride | N/A | 102 | 0.86 | Purity ≥ 98.0% | N/A |
| 16032125/16031116 | Methoxybenzene | N/A | 946 | 8.02 | Purity ≥ 99% | N/A |
| | (anisole) | | | | | |
| 17051771 | Et₃N | N/A | 47 | 0.40 | Purity ≥ 99.0% | N/A |
| | | | | | KF ≤ 0.2% | |
| C15050794-G17601 | 12% KHCO₃ | N/A | 1296 | 10.98 | Assay ≥ 99.0 | N/A |
| | | | | | % | |
| 170721/170919 | 25% NaCl aq. | N/A | 1404 | 11.90 | N/A | N/A |
| N/A | THF | N/A | 689 | 5.84 | Purity ≥ 99.8% | N/A |
| | | | | | KF ≤ 0.05% | |
| 170920/21074 | Process water | N/A | 804 | 6.81 | pH 6.5-8.5 | N/A |

### Plant Result for Preparation of C15050794-G

| | Starting | | | | | |
|---|---|---|---|---|---|---|
| | Material | | | | | |
| | (corrected | | Purity | Purity | | |
| Batch No. | by assay) | Product | (HPLC Area) | (w/w assay) | e.e. | Yield |
| C15050794-G17601 | 118.35 kg | 1243.4 kg | 92.8% | 12.6% | 96.6% | 87% |
| C15050794-G17602 | 117.35 kg | 1214.6 kg | 93.3% | 12.2% | 97.5% | 83% |

### Equipment for Preparation of C15050794-G17601-G17602

| Equip. Name | MBR Code | Process Requirement | Equip. Code |
|---|---|---|---|
| Reactor | R1 | GL/3000L | R210303 |
| | R2 | GL/3000L | R210103 |
| Pump | P1 | SS | P630040 |
| Peristaltic pump | P2 | SS | P636005 |
| Pump | P3 | SS | P634004 |
| Tank | T1 | HDPE | T12074 |
| | T2 | HDPE | T12046 |
| | T3 | HDPE | T12102 |
| | T4 | HDPE | T12101 |
| | T5 | HDPE | T12103 |
| | T6 | HDPE | T12105 |
| | T7 | HDPE | T12040 |
| | T8 | HDPE | T21063 |

### Detailed Process Description of C15050794-G

| | | G17601 | | G17602 | |
|---|---|---|---|---|---|
| Operation | | (1× = 118) | | (1× = 118) | |
| 1. | Charge process water (15.0-22.0×) | 2360 | kg | N/A | |
| | into R210103 | | | | |
| 2. | Adjust R210103 to 25-35° C. | 27.2° | C. | N/A | |
| 3. | Charge KHCO₃ (2.0-3.0×) into | 240 | kg | N/A | |
| | R210103 | | | | |
| 4. | Stir R210303 at 25-35° C. for | 40 | min | N/A | |
| | NLT 0.5 h | | | | |
| 5. | Load the material into tank | | | N/A | |
| 6. | Charge THF into R210303 | 323 | L | 323 | L |
| 7. | Heat R210303 to 60-70° C., | 64.2° | C. | 61.2° | C. |
| | distillate for 15-30 min | | | | |
| 8. | Reflux R210303 for 15-30 min | 30 | min | 30 | min |
| 9. | Adjust R210103 to 20-30° C. | 28.8° | C. | 26.1° | C. |
| 10. | Load the material into drums | | | | |
| 11. | Adjust R210303 to 100-110° C. | 104.9° | C. | 105.0° | C. |
| 12. | Dry R210303 for 1-2 h at | 1 | h | 2 | h |
| | 100-110° C. | | | | |
| 13. | Adjust R210303 to 20-40° C. | 34.7° | C. | 35.4° | C. |
| 14. | Charge CDI (0.67-0.80×) into | 93.3 | kg | 92.3 | kg |
| | R210303 | | | | |
| 15. | Charge anisole (6.8-9.0×) into | 840 | kg | 837 | kg |
| | R210303 | | | | |
| 16. | Adjust R210303 to −5-5° C. | −2.3° | C. | −1.2° | C. |
| 17. | Charge C15050794-SM5 | 90 | kg | 88.9 | kg |
| | (0.67-0.77×) into R210303 | | | | |
| 18. | Charge anisole into R210303 | 50 | kg | 41 | kg |
| 19. | Stir R210303 at −5-5° C. for 1-3 h | 2 | h | 2 | h |
| 20. | Charge TEA (0.36-0.42×) into | 48.2 | kg | 47 | kg |
| | R210303 | | | | |
| 21. | Stir R210303 at −5-5° C. for | 20 | h | 20 | h |
| | 10-20 h | | | | |
| 22. | Charge C15050794-SM6 | 118 | kg | 117 | kg |
| | (0.99-1.01×) into R210303 | | | | |
| 23. | Charge anisole into R210303 | 50 | kg | 48 | kg |
| 24. | Adjust R210303 to 35-45° C. and | 7 h 35 min | | 8 | h |
| | stir for 5-15 h | | | | |
| 25. | IPC: Purity of F (Spec.: FIO), | F % = 65.4%, | | F % = 65.8%, | |
| | SM6/F ≤1.0% | SM6/F = 0.1% | | SM6/F = 0.2% | |
| 26. | Adjust R210303 to −5-5° C. | −2.7° | C. | 0.7° | C. |
| 27. | Charge oxalyl chloride | 109 | kg | 102 | kg |
| | (0.76-1.05×) into R210303 | | | | |
| 28. | Charge anisole into R210303 | 22 | kg | 20 | kg |
| 29. | Stir R210303 for 1-3 h | 1 | h | 1 | h |
| 30. | IPC: Purity of G (Spec.: FIO), | G % = 76.5%, | | G % = 74.3%, | |
| | F/G ≤1.5% | F/G = 0.5% | | F/G = 0.8% | |
| 31. | Charge 12% KHCO₃ (8-12×) of | 1300 | kg | 1296 | kg |
| | step 5 into R210303 at −5-10° C. | | | | |
| 32. | Adjust R210303 to 15-25° C. and | 1 | h | 1 | h |
| | stir for 30-60 min | | | | |
| 33. | Stand for 30-60 min | 1 | h | 1 | h |
| 34. | Transfer the aqueous layer into | | | | |
| | tank | | | | |
| 35. | IPC: residual of G in aqueous | 0.2% | | 0.01% | |
| | layer (Spec.: FIO) | | | | |
| 36. | Charge 25% NaCl (4-5×) into | 590 | kg | 580 | kg |
| | R210303 | | | | |
| 37. | Charge process water (6-7×) into | 822 | kg | 804 | kg |
| | R210303 | | | | |
| 38. | Adjust R210303 to 15-25° C. and | 1 | h | 1 | h |
| | stir for 30-60 min | | | | |
| 39. | Stand for 30-60 min | 1 | h | 1 | h |
| 40. | Transfer the aqueous layer into | | | | |
| | tank | | | | |
| 41. | Charge 25% NaCl (10-12×) into | 1412 | kg | 1404 | kg |
| | R210303 | | | | |
| 42. | Adjust R210303 to 15-25° C. and | 1 | h | 1 | h |
| | stir for 30-60 min | | | | |
| 43. | Stand for 30-60 min | 1 | h | 1 | h |
| 44. | Transfer the aqueous layer into | | | | |
| | tank | | | | |
| 45. | IPC: residual of G in aqueous | N.D. | | 0.01% | |
| | layer (Spec.: FIO) | | | | |
| 46. | Charge THF into R210303 | 472 | L | 452 | L |
| 47. | Concentrate R210303 mixture to | 27.5° | C. | 25.5° | C. |
| | 1062-1534 L at ≤45° C. | | | | |
| 48. | Adjust R210303 to 20-30° C. | 27.3° | C. | 25.4° | C. |
| 49. | IPC: KF ≤0.10% | 0.03% | | 0.03% | |
| 50. | Adjust R210303 to 10-30° C. | 23.6° | C. | 25.7° | C. |

| | | | |
|---|---|---|---|
| 51. | Load the material into drum | Total weight: | Total weight: |
| | (C15050794-G anisole solution) | 1243.4 kg | 1214.6 kg |
| 52. | IPC: G % (Spec.: FIO), assay of | G % = 92.8%, | G % = 93.3%, |
| | G % (Spec.: FIO), e.e. % of G | assay of | assay of |
| | (Spec.: FIO) | G % = 12.6%, | G % = 12.2%, |
| | | e.e. % of | e.e. % of |
| | | G = 96.6% | G = 97.5% |

### C15050794-G (Example 1)

MS calcd C18 H29 N3 O6 [M+Na]+: 406.2, Found: 406.4, 1H NMR (300 MHz, CDCl₃) γ ppm 4.50 (br s, 1H), 4.23-4.01 (m, 4H), 3.96 (dd, J=4.7, 11.2 Hz, 1H), 3.66 (s, 2H), 3.01 (dt, J=3.8, 12.8 Hz, 1H), 2.81-2.59 (m, 2H), 1.55-1.42 (m, 9H), 1.37-1.23 (m, 6H), 1.21 (s, 6H)

### Example 2

### Preparation of C15050794-K (Example 2)

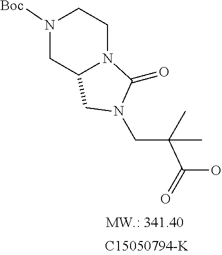 [SMILES: CC(C)(C)OC(=O)N1CCN2C(=O)N(CC(C)(C)C(=O)O)C[C@@H]2C1]

The title compound was prepared according to following scheme:

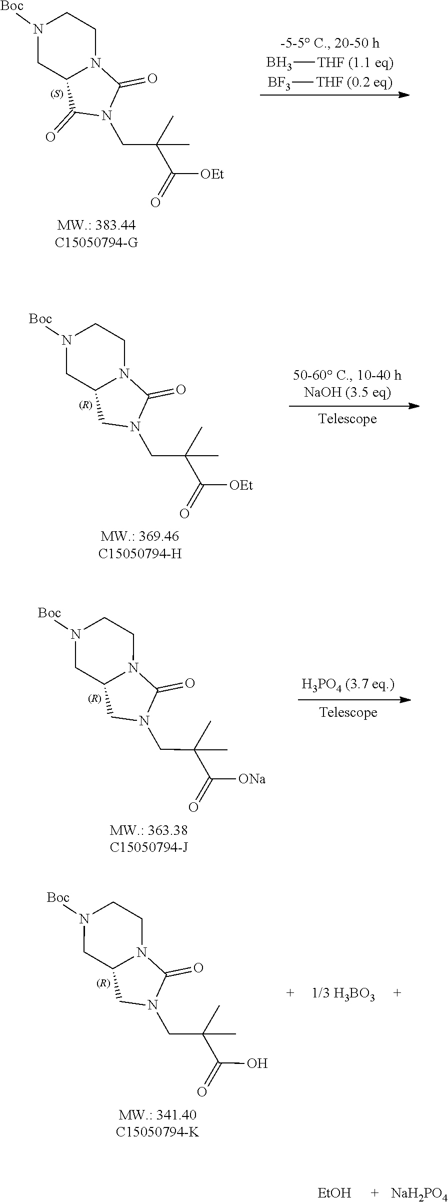

Production of C15050794-K was carried out in two batches. For C15050794-K17601, 56.75 kg (purity: 100.0%, assay: 100.0%, e.e. %: 99.2%) and 36.70 kg (purity: 100.0%, assay: 99.5%, e.e. %: 99.1%) of C15050794-K was obtained from 1239.0 kg of C15050794-G anisole solution (assay: 12.60%) in 67% yield. For C15050794-K17602, 54.45 kg (purity: 100.0%, assay: 98.6%, e.e. %: 99.4%) and 50.05 kg (purity: 100.0%, assay: 99.6%, e.e. %: 99.4%) of C15050794-K was obtained from 1214.6 kg of C15050794-G anisole solution (assay: 12.20%) in 78% yield. The details are summarized in table below.

### Raw Materials for Preparation of C15050794-K17601

| | | | Quantity | Rel Wt/Vol | | |
|---|---|---|---|---|---|---|
| Batch No. | Material | MW | (kg) | (1X = 156.0 kg) | Spec | Eq. |
| C15050794-G17601 | C15050794-G | 383.44 | 156.1 | 1.00 | Assay = 12.60% | 1.00 |
| 17091163 | BF₃-THF | 139.91 | 13 | 0.08 | Assay ≥ 45% | 0.23 |
| 17092868 | BF₃-THF (1 M) | 85.94 | 463 | 2.97 | Conc. = 0.95 M~1.10 M | 1.3 |
| PC00637-125-K | C15050794-K Seed | 341.4 | 0.25 | 0.00 | N/A | N/A |
| 17071961 | Na₂CO₃ | N/A | 45 | 0.29 | Assay = 98%~101% | N/A |
| 171003/171027B | 25% NaCl solution | N/A | 1350 | 8.65 | N/A | N/A |
| 16071562 | NaOH | N/A | 65.7 | 0.42 | Assay ≥ 98% | N/A |
| 171031 | MTBE | N/A | 402 | 2.58 | Purity ≥ 98.0% | N/A |
| | | | | | KF ≤ 0.1% | |
| 171031 | MeOH | N/A | 400 | 2.56 | Purity ≥ 99.5% | N/A |
| | | | | | KF ≤ 0.10% | |
| 17091862 | H₃PO₄ | N/A | 200.1 | 1.28 | Assay ≥ 85.0% | N/A |
| 171028/PW-21074, | Process water | N/A | 4740 | 30.38 | pH = 6.5-8.5 | N/A |
| 171029/PW-21073, | | | | | | |
| 171030/PW-21079, | | | | | | |
| 171101/PW-21079, | | | | | | |
| 171102/PW-21079, | | | | | | |
| 171104/PW-21074, | | | | | | |
| 171105/PW-21074 | | | | | | |
| 171027/171029 | THF | N/A | 324 | 2.08 | N/A | N/A |

### Raw Materials for Preparation of C15050794-K17602

| | | | Quantity | Rel Wt/Vol | | |
|---|---|---|---|---|---|---|
| Batch No. | Material | MW | (kg) | (1X = 148.0 kg) | Spec | Eq. |
| C15050794-G17602 | C15050794-G | 383.44 | 148.2 | 1.00 | Assay = 12.20% | 1.00 |
| 17091163 | BF₃-THF | 139.91 | 12.5 | 0.08 | Assay ≥ 45% | 0.23 |
| 17092868 | BF₃-THF (1 M) | 85.94 | 460.2 | 3.11 | Conc.= 0.95 M~1.10 M | 1.3 |
| PC00637-125-K | C15050794-K | 341.4 | 0.31 | 0.00 | N/A | N/A |
| | Seed | | | | | |
| 17071961/17091467 | Na₂CO₃ | N/A | 45 | 0.30 | Assay = 98%~101% | N/A |
| 171027B/171027A | 25% NaCl | N/A | 1356 | 9.16 | N/A | N/A |
| | solution | | | | | |
| 16071562 | NaOH | N/A | 65 | 0.44 | Assay ≥ 98% | N/A |
| 171104 | MTBE | N/A | 402 | 2.72 | Purity ≥ 98.0% | N/A |
| | | | | | KF ≤ 0.1% | |
| 171104/171107 | MeOH | N/A | 542 | 3.66 | Purity ≥ 99.5% | N/A |
| | | | | | KF ≤ 0.10% | |
| 17091862/17061610 | H₃PO₄ | N/A | 204.9 | 1.38 | Assay ≥ 85.0% | N/A |
| 171031/PW-21074, | Process water | N/A | 5410 | 36.55 | pH = 6.5-8.5 | N/A |
| 171101/PW-21074, | | | | | | |
| 171103/PW-21079, | | | | | | |
| 171104/PW-21079, | | | | | | |
| 171105/PW-21077, | | | | | | |
| 171106/PW-21039, | | | | | | |
| 171107/PW-21039 | | | | | | |
| 171027/171029 | THF | N/A | 234 | 1.58 | N/A | N/A |

### Plant Result for Preparation of C15050794-K

| | Starting | | | | | |
|---|---|---|---|---|---|---|
| | Material | | | | | |
| | (corrected | | Purity | Purity | | |
| Batch No. | by assay) | Product | (HPLC Area) | (w/w assay) | e.e | Yield |
| C15050794-K17601 | 156.11 kg | 56.75 kg | 100.0% | 100.0% | 99.2% | 67% |
| | | 36.70 kg | 100.0% | 99.5% | 99.1% | |
| C15050794-K17602 | 148.18 kg | 54.45 kg | 100.0% | 98.6% | 99.4% | 78% |
| | | 50.05 kg | 100.0% | 99.6% | 99.4% | |

### Equipment for Preparation of C15050794-K17601-K17602

| Equip. Name | MBR Code | Process Requirement | Equip. Code |
|---|---|---|---|
| Reactor | R1 | GL/3000L | R210302 |
| | R2 | GL/5000L | R210304 |
| | R3 | SS316L/3000L | R210403 |
| Tank | T1 | HDPE | T631121 |
| | T2 | HDPE | T631134 |
| | T3 | HDPE | T631158 |
| | T4 | SS | T630013 |
| | T5 | SS | T630018 |
| | T6 | HDPE | T631159 |
| | T7 | HDPE | T631166 |
| | V1 | GL | V2104C |
| | V2 | GL | V2104B |
| | V3 | GL | V2103A |
| Pump | P1 | PP | P634008 |
| | P2 | PP | P634005 |
| | P3 | SS | P630056 |
| Bag filter | Fb1 | SS | Fb630002 |
| Centrifuge | M1 | TI/HL | M210302 |
| | M2 | TI/HL | M210101 |
| Mother liquor tank | MV1 | GL | MV210302 |
| | MV2 | GL | MV210101 |
| Dryer | D1 | SS, Tray | D211001 |
| | D2 | SS, Tray | D211005 |

### Detailed Process Description of C15050794-K

| | | K17601 | | K17602 | |
|---|---|---|---|---|---|
| Operation | | (1× = 156.0 kg) | | (1× = 148.0 kg) | |
| 53. | Charge C15050794-G | 156.1 | kg | 148.2 | kg |
| | (1.00× ± 0.01×) anisole | | | | |
| | solution into R210302 | | | | |
| 54. | Charge THF (5-15 kg) into | 10 | kg | 8 | kg |
| | R210302 | | | | |
| 55. | Adjust R210302 to −10-5° C. | −5.1° | C. | −4.8° | C. |
| 56. | Charge BH₃—THF (1M) | 88.0 | kg | 87.2 | kg |
| | (0.3-2.0×) into R210302 | | | | |
| 57. | Charge THF (5-15 kg) into | 12 | kg | 14 | kg |
| | R210302 | | | | |
| 58. | Charge BH₃—THF | 13.0 | kg | 12.5 | kg |
| | (0.065-0.106×) into R210302 | | | | |
| 59. | Charge THF (5-15 kg) into | 8 | kg | 6 | kg |
| | R210302 | | | | |
| 60. | Charge BH₃—THF (1M) | 375 | kg | 373 | kg |
| | (1.5-3.5×) into R210302 | | | | |
| 61. | Charge THF (5-15 kg) into | 6 | kg | 8 | kg |
| | R210302 | | | | |
| 62. | Adjust R210302 to 5-5° C. | −1.6° | C. | −1.4° | C. |
| 63. | Stir R210302 for 20-50 h | 30 | h | 28 | h |
| 64. | IPC: G/H % (Spec. ≤3%), | G/H % = 1%, | | G/H % = 1%, | |
| | purity of H % (Spec.: FIO) | H % = 81.4% | | H % = 82.7% | |
| 65. | Adjust R210302 to −10-0° C. | −0.2° | C. | −4.6° | C. |
| 66. | Charge Na₂CO₃ (0.16-0.48×) | 45.0 | kg | 45.0 | kg |
| | into R210304 | | | | |
| 67. | Charge process water (9-13×) | 1754 | kg | 1796 | kg |
| | into R210304 | | | | |
| 68. | Adjust R210302 to 25-35° C., | 30.8° | C. | 26.8° | C. |
| | stir NLT 0.5 h | | | | |
| 69. | Adjust R210302 to 0-20° C. | 8.1° | C. | 8.1° | C. |
| 70. | Charge C15050794-H | | | | |
| | solution into R210304 in | | | | |
| | portions | | | | |
| 71. | Charge THF (20-50 kg) into | 46 | kg | 30 | kg |
| | R210302 | | | | |
| 72. | Charge the THF solution | | | | |
| | above into R210304 | | | | |
| 73. | Stir R210304 for 0.5-1.0 h | 40 | min | 45 | min |
| 74. | Adjust R210304 to 20-30° C., | 21.6° | C. | 23.5° | C. |
| | stir for 0.5-1.0 h and stand | | | | |
| | for 1-5 h | | | | |
| 75. | Transfer R210304 aqueous | | | | |
| | layer into T631121 and | | | | |
| | T631134 | | | | |
| 76. | IPC: Residual of H in aqueous | Residual of | | Residual of | |
| | layer (%, w/w) (Spec.: FIO) | H in aqueous | | H in aqueous | |
| | | layer | | layer | |
| | | (%, w/w) = | | (%, w/w) = | |
| | | 0.02% | | 0.02% | |
| 77. | Charge 25% NaCl solution | 450 | kg | 454 | kg |
| | (2.4-4.7×) into R210304 | | | | |
| 78. | Charge process water | 450 | kg | 436 | kg |
| | (2.4-4.7×) into R210304 | | | | |
| 79. | Adjust R210304 to 20-30° C., | 24.8° | C. | 24.5° | C. |
| | stir for 0.5-1.5 h and stand | | | | |
| | for 2-4 h | | | | |
| 80. | Transfer R210304 aqueous | | | | |
| | layer into V2103A and | | | | |
| | T631158, label material tag | | | | |
| 81. | Charge 25% NaCl solution | 900 | kg | 902 | kg |
| | (4.7-8.2×) into R210304 | | | | |
| 82. | Adjust R210304 to 20-30° C., | 24.2° | C. | 24.0° | C. |
| | stir for 0.5-1.5 h and stand | | | | |
| | for 1-3 h | | | | |
| 83. | Transfer R210304 aqueous | | | | |
| | layer into V2103A and | | | | |
| | T631158, label material tag | | | | |
| 84. | IPC: KF ≤3.0% | KF = 1.1% | | KF = 1.0% | |
| 85. | IPC: Purity of H % | Purity of | | Purity of | |
| | (Spec.: FIO), assay of H | H % = 85.8%, | | H % = 86.0%, | |
| | (%, w/w) (Spec.: FIO) | assay of H | | assay of H | |
| | | (%, w/w) = | | (%, w/w) = | |
| | | 7.9% | | 8.0% | |
| 86. | Transfer R210304 organic | | | | |
| | layer into T630013 and | | | | |
| | t630018 and label material | | | | |
| | tag | | | | |
| 87. | IPC: Residual of H in | Residual of | | Residual of | |
| | aqueous layer (%, w/w): FIO | H in aqueous | | H in aqueous | |
| | | layer | | layer | |
| | | (%, w/w) = | | (%, w/w) = | |
| | | 0.003% | | 0.001% | |
| 88. | Load the material in V2103A | | | | |
| | into iron drums and label | | | | |
| | material tag | | | | |
| 89. | Transfer tank organic layer | | | | |
| | into R210403 | | | | |
| 90. | Charge THF (5-30 kg) into | 30 | kg | 30 | kg |
| | R210403 | | | | |
| 91. | Charge process water | 14 | kg | 20 | kg |
| | (0.0-0.3×) into R210403 | | | | |
| 92. | Charge THF (0-4×) into | 166 | kg | 168 | kg |
| | R210403 | | | | |
| 93. | Charge NaOH (0.35-0.59×) | 65.7 | kg | 65.0 | kg |
| | in portions into R210403 | | | | |
| 94. | Adjust R210403 to 50-60° C. | 33 | h | 34.5 | h |
| | and stir for 10-40 h | | | | |
| 95. | Adjust R210403 to 30-40° C. | 39.8° | C. | 38.6° | C. |
| 96. | IPC: Residual of H (%, w/w) | Residual of H | | Residual of H | |
| | (Spec. ≤0.15%) | (%, w/w) = | | (%, w/w) = | |
| | | 0.001% | | 0.001% | |
| 97. | Adjust R210403 to 20-30° C. | 26.1° | C. | 27.1° | C. |
| 98. | Charge process water into | 1202 | kg | 1106 | kg |
| | R210403 | | | | |
| 99. | Stir R210403 for 0.5-1.5 h | 1.5 | h | 1.0 | h |
| 100. | Stand R210403 for 1-3 h | 3 | h | 3 | h |
| 101. | Transfer R210403 aqueous | | | | |
| | layer into T631159 and | | | | |
| | T631166, label material tag | | | | |
| 102. | Transfer R210403 organic | | | | |
| | layer into V2104C and | | | | |
| | V2104B, label material tag | | | | |
| 103. | Transfer T631159 and | | | | |
| | T631166 aqueous layer into | | | | |
| | R210403 | | | | |
| 104. | Charge process water | 590 | kg | 682 | kg |
| | (1.2-4.7×) into T631159 and | | | | |
| | T631166 | | | | |
| 105. | Transfer aqueous layer in | | | | |
| | T631159/T631166 into | | | | |
| | R210403 | | | | |
| 106. | Charge MTBE (1.8-4.1×) | 402 | kg | 402 | kg |
| | into R210403 | | | | |
| 107. | Adjust R210403 to 20-30° C., | 23.6° | C. | 23.8° | C. |
| | stir for 0.5-1.5 h | | | | |
| 108. | Stand R210403 for 1-3 h | 2 | h | 2 | h |
| 109. | Transfer R210403 aqueous | | | | |
| | layer into T631159 and | | | | |
| | T631166 | | | | |
| 110. | Transfer R210403 organic | | | | |
| | layer into V2104C and | | | | |
| | V2104B | | | | |
| 111. | IPC: Purity of K % in | Purity of K % | | Purity of K % | |
| | aqueous layer (Spec.: FIO) | in aqueous | | in aqueous | |
| | | layer = 99.7% | | layer = 99.1% | |
| 112. | IPC: Residual of K (%, w/w) | Residual of K | | Residual of K | |
| | in organic layer (Spec.: FIO) | (%, w/w) in | | (%, w/w) in | |
| | | organic | | organic | |
| | | layer = 5.3% | | layer = N.D. | |
| 113. | Load the material in | | | | |
| | V2104CA/2104B into iron | | | | |
| | drums and label material tag | | | | |
| 114. | Charge THF (15-50 kg) into | 46 | kg | MeOH: 150 kg | |
| | R210403 | | | | |
| 115. | Load the material in R210403 | | | | |
| | into iron drums and label | | | | |
| | material tag | | | | |
| 116. | Transfer T631159 and | | | | |
| | T631166 aqueous layer into | | | | |
| | R210302 | | | | |
| 117. | Charge MeOH (0.0-3.5×) into | 400 | kg | 392 | kg |
| | R2103202 | | | | |
| 118. | Adjust R210302 to 20-40° C. | 25.9° | C. | 26.2° | C. |
| 119. | Charge H₃PO₄ (0.77-1.28×) | 160 | kg | 160 | kg |
| | into R210302 | | | | |
| 120. | Charge C15050794-K seed | 0.250 | kg | 0.310 | kg |
| | (0.0001-0.0100×) into | | | | |
| | R210302 | | | | |
| 121. | Adjust R210302 to 30-40° C. | 31.1° | C. | 32.3° | C. |
| | and stir for 1-2 h | | | | |
| 122. | Charge H₃PO₄ (0.19-0.32×) | 40.1 | kg | 44.9 | kg |
| | into R210302 | | | | |
| 123. | Adjust R210302 to 15-25° C. | 24.9° | C. | 24.7° | C. |
| | and stir for 1-3 h | | | | |
| 124. | IPC: Residual of K (%, w/w) | Residual of K | | Residual of K | |
| | in the filtrate supernatant | (%, w/w) = | | (%, w/w) = | |
| | (Spec. ≤0.25%) | 0.18% | | 0.16% | |
| 125. | Spread centrifuge bag in | | | | |
| | M210302 | | | | |
| 126. | Transfer R210302 material | | | | |
| | in portions into M210302 | | | | |
| | for centrifuge. During the | | | | |
| | centrifuging, maintain the | | | | |
| | reactor temperature at | | | | |
| | 15-25° C. and agitation | | | | |
| 127. | Charge process water | 34 | kg | 178 | kg |
| | (0.19-1.5×) to rinse the wet | | | | |
| | cake | | | | |
| 128. | Still centrifuge and blow, | | | | |
| | R210302 for at least 10 min | | | | |
| 129. | Load solid according to | | | | |
| | instruction of step 147 | | | | |
| 130. | Charge process water | 140 | kg | 180 | kg |
| | (0.5-1.5×) to rinse the wet | | | | |
| | cake | | | | |
| 131. | Still centrifuge and blow, | | | | |
| | R210302 for at least 10 min | | | | |
| 132. | Load solid according to | | | | |
| | instruction of step 147 | | | | |
| 133. | Charge process water | 140 | kg | 180 | kg |
| | (0.5-1.5×) to rinse the wet | | | | |
| | cake | | | | |
| 134. | Still centrifuge and blow, | | | | |
| | R210302 for at least 10 min | | | | |
| 135. | Load solid according to | | | | |
| | instruction of step 147 | | | | |
| 136. | Charge process water | 164 | kg | 832 | kg |
| | (0.5-1.5×) to rinse the wet | | | | |
| | cake | | | | |
| 137. | Still centrifuge and blow, | | | | |
| | R210302 for at least 10 min | | | | |
| 138. | Load solid according to | | | | |
| | instruction of step 147 | | | | |
| 139. | Charge process water | 162 | kg | N/A | |
| | (0.5-1.5×) to rinse the wet | | | | |
| | cake | | | | |
| 140. | Still centrifuge and blow, | | | N/A | |
| | R210302 for at least 10 min | | | | |
| 141. | Load solid according to | | | N/A | |
| | instruction of step 147 | | | | |
| 142. | Charge process water | 230 | kg | N/A | |
| | (0.5-1.5×) to rinse the wet | | | | |
| | cake | | | | |
| 143. | Still centrifuge and blow, | | | N/A | |
| | R210302 for at least 10 min | | | | |
| 144. | Load solid according to | | | N/A | |
| | instruction of step 147 | | | | |
| 145. | Spread centrifuge bag in | | | N/A | |
| | M210101 | | | | |
| 146. | Transfer R210302 material | | | N/A | |
| | in portions into M210101 | | | | |
| | for centrifuging. During the | | | | |
| | centrifuging, maintain the | | | | |
| | reactor temperature at | | | | |
| | 15-25° C. and agitation | | | | |
| 147. | Load solid into fiber drum | Total weight: | | Total weight: | |
| | lined with PE bags and label | 153.35 kg | | 172.65 kg | |
| | material tag | | | | |
| 148. | IPC: Purity of the wet cake | Purity of the | | Purity of the | |
| | % (Spec. ≥98.0%) e.e. % of | wet cake | | wet cake | |
| | the wet cake (Spec. ≥295.0%) | (%) = | | (%) = | |
| | | 100.0% e.e. | | 100.0% e.e. | |
| | | % of the wet | | % of the wet | |
| | | cake = 99.1% | | cake = 99.2% | |
| 149. | IPC: Residua of K (%, w/w) | Residua; of K | | Residua; of K | |
| | (Spec.: FIO) | (%, w/w) = | | (%, w/w) = | |
| | | 0.2% | | 0.2% | |
| 150. | IPC: Residua of K (%, w/w) | Residua; of K | | N/A | |
| | in organic layer (Spec.: FIO) | (%, w/w) = | | | |
| | | N.D. | | | |
| 151. | Dry the wet cake for two | 53.64° | C. | 57.73° | C. |
| | batches. For the first batch: | | | | |
| | put C15050794-K wet cake | | | | |
| | into drying bag, then put the | | | | |
| | bag into D211001, adjust | | | | |
| | jacket temperature to | | | | |
| | 50-60° C. | | | | |
| 152. | Dry D211001 under reduces | 19 | h | 20 | h |
| | pressure at 50-60° C. for | | | | |
| | 10-20 h | | | | |
| 153. | Dry D211001 under reduces | 22 | h | 20 | h |
| | pressure at 60-70° C. for | | | | |
| | 10-20 h | | | | |
| 154. | IPC: KF ≤1.0% | KF = 4.3% | | KF = 0.1% | |
| 155. | Dry D211001 under reduces | 22 | h | N/A | |
| | pressure at 50-70° C. for | | | | |
| | 10-20 h | | | | |
| 156. | IPC: KF ≤1.0% | KF = 0.2% | | N/A | |
| 157. | Adjust D211001 to 20-30° C. | 29.51° | C. | 25.83° | C. |
| 158. | Hold D211001 for 20-40 min | 22 | min | 34 | min |
| 159. | IPC: Purity of K % | Purity of | | Purity of | |
| | (Spec. ≥98.0%), assay of K | K % = | | K % = | |
| | (%, w/w) (Spec.: FIO), e.e. | 100.0%, | | 100.0%, | |
| | % of K (Spec. ≥95.0%) | assay of K | | assay of K | |
| | | (%, w/w) = | | (%, w/w) = | |
| | | 100.0%, | | 98.6%, | |
| | | e.e. % of | | e.e. % of | |
| | | K = 99.2% | | K = 99.4% | |
| 160. | Calculate the net wet | Total weight: | | Total weight: | |
| | | 56.75 kg | | 54.45 kg | |
| 161. | The second batch: put | 55.33° | C. | 56.65° | C. |
| | C15050794-K wet cake into | | | | |
| | drying bag, then put the bag | | | | |
| | into D211001, adjust jacket | | | | |
| | temperature to 50-60° C. | | | | |
| 162. | Dry D211001 under reduces | 20 | h | 20 | h |
| | pressure at 50-60° C. for | | | | |
| | 10-20 h | | | | |
| 163. | Dry D211001 under reduced | 20 | h | 20 | h |
| | pressure at 60-70° C. for | | | | |
| | 10-20 h | | | | |
| 164. | IPC: KF ≤1.0% | KF = 0.3% | | KF = 0.2% | |
| 165. | Adjust R211001 to 20-30° C. | 27.28° | C. | 26.09° | C. |
| 166. | Hold D211001 for 20-40 min | 25 | min | 37 | min |

| | | | |
|---|---|---|---|
| 167. | IPC: Purity of K % | Purity of | Purity of |
| | (Spec. ≥98.0%), assay of K | K % = | K % = |
| | (%, w/w) (Spec.: FIO), e.e. | 100.0%, | 100.0%, |
| | % of K (Spec. ≥95.0%) | assay of K | assay of K |
| | | (%, w/w) = | (%, w/w) = |
| | | 99.5%, | 99.6%, |
| | | e.e. % of | e.e. % of |
| | | K = 99.1% | K = 99.4% |
| 168. | Calculate the net wet | Total weight: | Total weight: |
| | | 36.70 kg | 50.05 kg |

### C15050794-K (Example 2)

HRMS calcd C16 H27 N3 O5 [M+H]+: 341.1951, Found: 341.1976, 1H NMR (600 MHz, CHLOROFORM-d) δ ppm 3.90-4.36 (m, 2H), 3.70-3.84 (m, 1H), 3.53-3.63 (m, 1H), 3.46-3.52 (m, 1H), 3.29-3.43 (m, 2H), 3.02 (dd,J=9.1, 4.7 Hz, 1H), 2.36-2.92 (m, 3H), 1.40-1.50 (m, 9H), 1.15-1.30 (m, 6H)

### Example 3

### Preparation of C15050794-SM2 (Example 3)

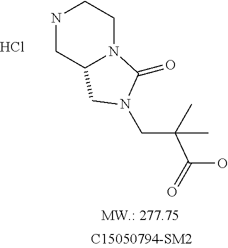 [SMILES: Cl ; CC(C)(CN1C[C@@H]2CNCCN2C1=O)C(=O)O]

The title compound was prepared according to following scheme:

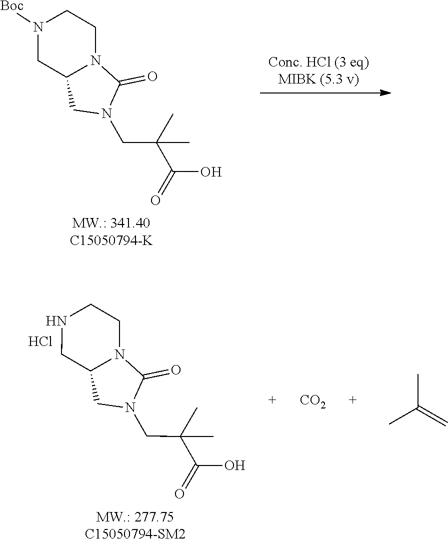

Production of C15050794-SM2 was carried out in one batch. For C15050794-SM2 17601, 157.25 kg of C15050794-SM2 was obtained from 197.20 kg of C15050794-K with 99.9% purity, 92.1% assay, 99.3% e.e. in 90% yield. The details are summarized in table below.

### Raw Materials for Preparation of C15050794-SM2 17601

| | | | | Rel | | |
|---|---|---|---|---|---|---|
| | | | Quantity | Wt/Vol | | |
| Batch No. | Material | MW | (kg) | (1X = 196 kg) | Spec | Eq. |
| C15050794- | C15050794-K | 341.4 | 197 | 1.01 | Assay = 100.0% | 1.0 |
| K17601A/C15050794- | | | | | Assay = 99.5% | |
| K17601B/C15050794- | | | | | Assay = 98.6% | |
| K17602A/C15050794- | | | | | Assay = 99.6% | |
| K17602B | | | | | | |
| PC00665-100-SM2 | C15050794-SM2 | 277.75 | 0.15 | 0.00 | Assay ≥ 78% | N/A |
| | Seed | | | | | |
| 17093067 | 35% HCI | N/A | 171 | 0.87 | Assay = 32%~39% | N/A |
| 171127 | Acetone | N/A | 528 | 2.69 | Purity ≥ 99.5% | N/A |
| | | | | | KF ≤ 0.3% | |
| 17091969 | MIBK | N/A | 951 | 4.85 | Purity ≥ 99.0% | N/A |
| | | | | | KF ≤ 0.1% | |

### Plant Result for Preparation of C15050794-SM2 17601

| | Starting | | | | | |
|---|---|---|---|---|---|---|
| | Material | | | | | |
| | (corrected | | Purity | Purity | | |
| Batch No. | by assay) | Product | (HPLC Area) | (w/w assay) | e.e. | Yield |
| C15050794-SM2 17601 | 197.20 kg | 157.25 kg | 99.9% | 92.1% | 99.3% | 90% |

### Equipment for Preparation of C15050794-SM2 17601

| Equip. Name | MBR Code | Process Requirement | Equip. Code |
|---|---|---|---|
| Reactor | R1 | GL/3000L | R210101 |
| Pump | P1 | PP/SS | P630058 |
| | P2 | PP | P634007 |
| Centrifuge | M1 | TI/HL | M210102 |
| Mother liquor tank | MV1 | GL | MV210102 |
| Dray | D1 | GL/SS, Double cone or SS, | D120206 |
| | | Single cone | |

### Detailed Process Description of C15050794-SM2 17601

| | | SM2 17601 | |
|---|---|---|---|
| Operation | | (1× = 196) | |
| 169. | Charge MIBK (4-5×) into R210101 | 901 | kg |
| 170. | Charge C15050794-K (0.99-1.01×) into | 6.00 | kg |
| | R210101 | | |
| 171. | Charge MIBK (20-50 kg) into R210101 | 50 | kg |
| 172. | Adjust R210101 to 20-30° C. | 22.9° | C. |
| 173. | Charge 35% HCl (0.80-0.92×) into R210101 | 171.0 | kg |
| 174. | Stir R210101 for 8-16 h | 16 | h |
| 175. | IPC: Residual of K (%, w/w) (Spec. ≤15%) | Residual of K | |
| | | (%, w/w) = | |
| | | 0.01% | |
| 176. | Adjust R210101 to 15-20° C. | 19.5° | C. |
| 177. | Concentrate R210101 mixture at ≤60° C. to | | |
| | 392-784 L | | |
| 178. | Adjust R210101 to 20-40° C. | 31.9° | C. |
| 179. | Charge acetone (4.0-5.0×) into R210101 | 971 | L |
| 180. | Concentrate R210101 mixture at ≤60° C. to | | |
| | 588-980 L | | |
| 181. | Adjust R210101 to 45-55° C. | 45.8° | C. |
| 182. | Charge acetone (4.0-5.0×) into R210101 | 971 | L |
| 183. | Charge C15050794-SM2 (0.0001-0.0010×) | 0.150 | kg |
| | crystal seed into R210101 | | |
| 184. | Charge acetone (20-50 kg) into R210101 | 36 | kg |
| 185. | Adjust R210101 to 50-60° C. | 54.4° | C. |
| 186. | Stir R210101 for 0.5-1 h | 1 | h |
| 187. | Adjust R210101 to 20-40° C. | 35.3° | C. |
| 188. | IPC: Residual of SM2 (%, w/w) | Residual of | |
| | (Spec. ≤0.7%), KF ≤3.5% | SM2 | |
| | | (%, w/w) = | |
| | | 0.2%, | |
| | | KF = 2.9% | |
| 189. | Adjust R210101 to 18-22° C. for over 3 h | 20.6° | C. |
| 190. | Stir R210101 for 1-3 h | 3 | h |
| 191. | Spread centrifuge bag in M210102 | | |
| 192. | Transfer R210101 material in portions into | | |
| | M210102 for centrifuging. During the | | |
| | centrifuging, maintain the reactor temperature | | |
| | at 18-22° C. and agitation | | |
| 193. | Charge acetone (1.3-5.0×) to rinse the wet cake | 70 | kg |
| 194. | Load solid according to instruction of step 205 | | |
| 195. | Charge acetone (1.3-5.0×) to rinse the wet cake | 74 | kg |
| 196. | Load solid according to instruction of step 205 | | |
| 197. | Charge acetone (1.3-5.0×) to rinse the wet cake | 78 | kg |
| 198. | Load solid according to instruction of step 205 | | |
| 199. | Charge acetone (1.3-5.0×) to rinse the wet cake | 68 | kg |
| 200. | Load solid according to instruction of step 205 | | |
| 201. | Charge acetone (1.3-5.0×) to rinse the wet cake | 70 | kg |
| 202. | Load solid according to instruction of step 205 | | |
| 203. | Charge acetone (1.3-5.0×) to rinse the wet cake | 132 | kg |
| 204. | Load solid according to instruction of step 205 | | |
| 205. | Load solid into fiber drum lined with double | Total weight: | |
| | PE bags and label material tag | 167.60 kg | |
| 206. | IPC: Purity of the wet cake % (Spec. ≥98.0%) | Purity of the | |
| | | wet cake | |
| | | % = 99.8% | |
| 207. | IPC: Residual of SM2 (%, w/w) (Spec.: FIO) | Residual | |
| | | of SM2 | |
| | | (%, w/w) = | |
| | | 0.1% | |
| 208. | Put the wet cake into D 120206 | | |
| 209. | Adjust D120206 to 30-40° C. | 40° | C. |
| 210. | Dry D120206 under reduced pressure at | 4 | h |
| | 30-40° C. for 3-5 h | | |
| 211. | Adjust D120206 to 40-50° C. | 43.3° | C. |
| 212. | Dry D120206 under reduced pressure at | 12 | h |
| | 40-50° C. for 7-15 h | | |
| 213. | IPC: KF ≤7% | KF = 4% | |
| 214. | Adjust D120206 to 20-30° C. | 29.7° | C. |

| | | |
|---|---|---|
| 215. | Hold D120206 for 1 h | |
| 216. | IPC: Assay of SM2 (%, w/w) (Spec.: FIO), | Assay of SM2 |
| | purity of SM2% (Spec. ≥98.0%), e.e. of | (%, w/w) = |
| | SM2% (Spec. ≥95.0%) | 92.1%, purity |
| | | of SM2% = |
| | | 99.9%, e.e. |
| | | of SM2% = |
| | | 99.3% |
| 217. | Calculate the net wet | Total weight: |
| | | 157.25 kg |

### C15050794-SM2 (Example 3):

1H NMR (600 MHz, DMSO-d6) δ ppm 12.10-12.59 (m, 1H), 9.32-9.78 (m, 2H), 3.85-3.95 (m, 1H), 3.75-3.76 (m, 1H), 3.68-3.76 (m, 1H), 3.41-3.47 (m, 1H), 3.23-3.27 (m, 1H), 3.15-3.18 (m, 1H), 3.13-3.30 (m, 2H), 3.13-3.17 (m, 1H), 3.00-3.06 (m, 1H), 2.69-2.79 (m, 1H), 2.66-2.75 (m, 1H), 1.08 (d, J=7.8 Hz, 6 H); HRMS calcd C11 H19 N3 03 [M+H]+: 241.1426, Found: 241.1429

### Example 4

### Preparation of ethyl 4-(3-fluoro-2-methyl-phenyl)-6-methyl-2-thiazol-2-yl-1,4-dihydropyrimidine-5-carboxylate (Example 4)

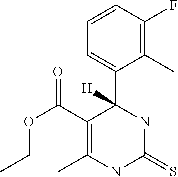 [SMILES: CCOC(=O)C1=C(C)NC(=S)N[C@H]1c1cccc(F)c1C]

The title compound was prepared according to following scheme:

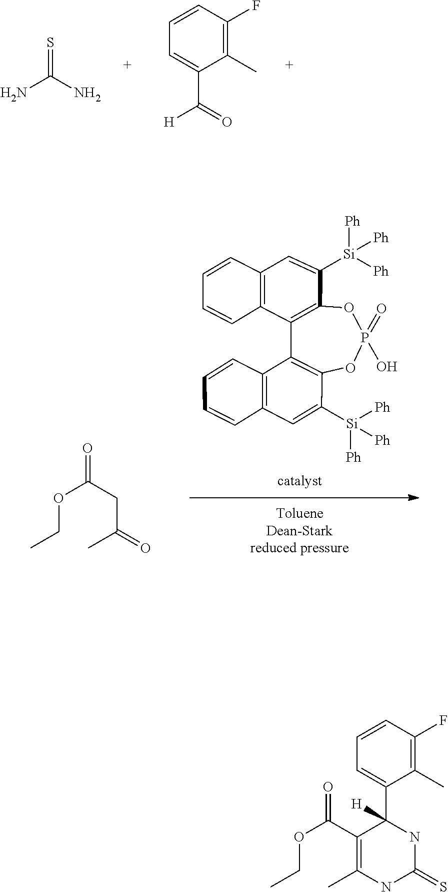

In a reactor configured for Dean-Stark water removal, a suspension was prepared from thiourea (12.73 g, 167.2 mmol, 1.05 equiv.), 3-fluoro-2-methyl-benzaldehyde (22.0 g, 159.3 mmol, 1.00 equiv.), and ethyl acetoacetate (24.87 g, 191.1 mmol, 1.20 equiv.), (R)-(−)-3,3′-Bis(triphenylsilyl)-1,1-binaphthyl-2,2′-diyl hydrogen-phosphate (1.38 g, 1.59 mmol, 0.01 equiv.) and toluene (76.1 g). This mixture was stirred at 80° C. jacket temperature under reduced pressure in order to achieve gentle reflux and Dean-Stark removal of the water generated during the reaction over 15-18 h. Upon reaction completion, the suspension was cooled to 15° C. and stirred for at least 2 h. The crystals were filtered, washed with pre-cooled toluene (26 g) and dried under reduced pressure at 50° C. The isolated yield was 40.6 g (82%) with 95% enantiopurity. 1H NMR (600 MHz, DMSO-d6) δ ppm 10.30 (m, 1H), 9.56 (br d, J=0.8 Hz, 1H), 7.23 (m, 1H), 7.07 (m, 1H), 7.02 (dd, J=8.1, 0.9 Hz, 1H), 5.43 (d, J=3.2 Hz, 1H), 3.92 (q, J=7.1 Hz, 2H), 2.33 (d, J=1.6 Hz, 3H), 2.32 (d, J=0.5 Hz, 3H), 1.00 (t, J=7.1 Hz, 3H) HRMS calcd C15 H17 N2 O2 S [M+H]+: 308.0995, Found: 308.1002

### Example 5

### Preparation of ethyl (4S)-4-(3-fluoro-2-methyl-phenyl)-6-methyl-2-oxo-3,4-dihydro-1H-pyrimidine-5-carboxylate (Example 5)

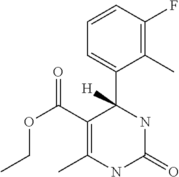 [SMILES: CCOC(=O)C1=C(C)NC(=O)N[C@H]1c1cccc(F)c1C]

The title compound was prepared according to following scheme:

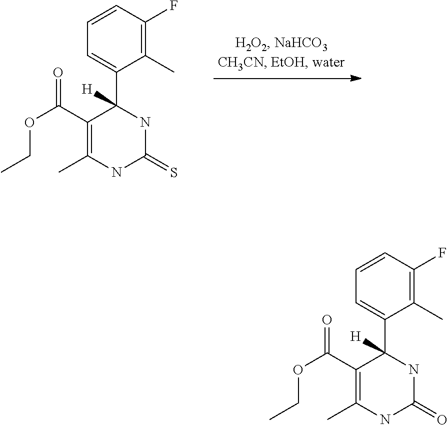

Ethyl (4S)-4-(3-fluoro-2-methyl-phenyl)-6-methyl-2-thioxo-3,4-dihydro-1H-pyrimidine-5-carboxylate (30 g, 97.3 mmol, 1.0 equiv.), suspended in acetonitrile (59.9 g), ethanol (58.95 g), sodium bicarbonate (32.79 g, 389.1 mmol, 4 equiv.) and water (390 g) was stirred at room temperature for 30 minutes. The suspension was cooled to 5-10° C. and the hydogen peroxide (3 wt % solution in water, 75.64 g, 778 mmol, 8 equiv.) was added over 4 h. Minimal effervescence was observed with this rate of addition. The resulting suspension was stirred for 15-18 h at 5-10° C. Upon reaction completion, water (150 g) was added and the suspension was warmed to 25° C. and stirred for another 5 h. The crystals were filtered, washed with two portions of 9:1 v/v water/acetonitrile (total 120 mL) and dried under reduced pressure at 50° C. The isolated yield was 25.8 g (90.8%), with assay approx. 92%. Chiral purity observed in the starting material was preserved.

To recrystallize this material, the crude solid (25.8 g) was dissolved in MeTHF (500 mL), polish filtered, and then partially concentrated under reduced pressure (jacket temperature 30° C.) to approx. 300 mL. n-Heptane (600 mL) was added over 30 minutes and the resulting white suspension was cooled to 10-15° C. (internal temperature), filtered and dried. The overall yield was 21.4 g (75.3%), with assay approx. 100%. Chiral purity was unchanged. 1H NMR (600 MHz, DMSO-d6) δ ppm 9.20 (d, J=1.3 Hz, 1H), 7.66 (t, J=2.3 Hz, 1H), 7.20 (m, 1H), 6.98-7.06 (m, 2H), 5.42 (d, J=2.6Hz, 1H), 3.89 (m, 2H), 2.30 (d, J=1.7 Hz, 3H), 2.29 (d, J=0.6 Hz, 3H), 0.99 (t, J=7.1 Hz, 3H); HRMS calcd C15 H17 N2 O3 [M+H]+: 239.1296, Found: 293.1301

### Example 6

### Preparation of ethyl (4S)-2-chloro-4-(3-fluoro-2-methyl-phenyl)-6-methyl-1,4-dihydropyrimidine-5-carboxylate (Example 6)

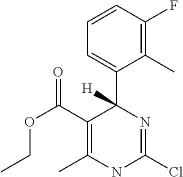 [SMILES: CCOC(=O)C1=C(C)NC(Cl)=N[C@H]1c1cccc(F)c1C]

The title compound was prepared according to following scheme:

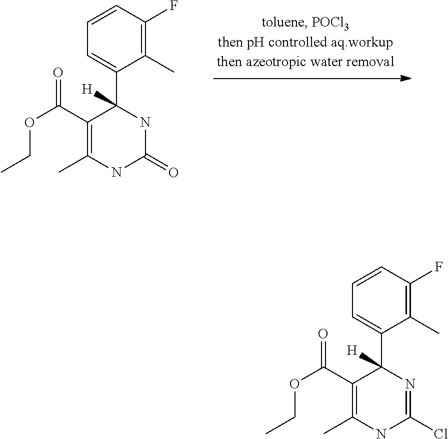

Ethyl (4S)-4-(3-fluoro-2-methyl-p henyl)-6-methyl-2-oxo-3, 4-di hydro-1H-pyrimidine-5-carboxylate (20 g, 68.4 mmol, 1.0 equiv., assay min 92%) was suspended in toluene (43.2 g) and phosphoryl chloride (34.47 g, 205.3 mmol, 3.0 eqiv.). Additional toluene (8.7 g) was used to rinse the addition funnel. The white suspension was heated to 100° C. (internal temperature) and a yellow solution was obtained after approx. 15 minutes, eventually becoming a red solution. The reaction was stirred for 24 h and then diluted with toluene (51.9 g) and cooled to 0° C. This solution was dosed over 60 min into second vessel containing vigorously stirring mixture of toluene (51.9 g) and K₂HPO₄ (5% w/w aqueous solution, 60.0 g) at 0° C. The quench vessel was maintained below 15° C. (internal temperature) and the pH maintained in the range 7.0-8.5 by variable rate co-dosing of KOH (48% w/w aqueous solution, 230.3 g). The addition rate of the KOH solution was continued beyond the reaction mixture dosing to maintain the pH range (end pH was approx. 7.8). The resulting biphasic mixture was warmed to 23° C. (jacket temperature) and stirred for 1 h. The lower aqueous layer was removed and the organic layer washed twice with K₂HPO₄ (5% w/w aqueous solution, 200 g total). The organic solution was polish filtered and the filter rinsed with toluene (17.3 g). The toluene solution was distilled under reduced pressure while maintaining 25° C. (jacket temperature), with replacement with fresh toluene until water-free, and to achieve a final volume of 200 mL. This 0.34 M solution of ethyl (4S)-2-chloro-4-(3-fluoro-2-methyl-phenyl)-6-methyl-1,4-dihydropyrimidine-5-carboxylate in toluene was used directly (uncorrected for assay). 1H NMR (600 MHz, DMSO-d6) δ ppm 9.81-10.33 (m, 1H), 7.16-7.28 (m, 1H), 7.05 (t,J=9.0 Hz, 1H), 7.00 (d,J=7.7 Hz, 1H), 5.74 (s, 1H), 3.91 (d,J=7.1 Hz, 2H), 2.24-2.38 (m, 6H), 0.98 (t,J=7.1 Hz, 3H); HRMS calcd C15 H16 Cl F N2 O2 [M+H]+: 310.0898, Found: 310.0884

### Example 7

### Preparation of bromo(thiazol-2-yl)zinc solution in THF (Example 7)

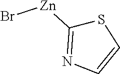

The title compound was prepared according to following scheme:

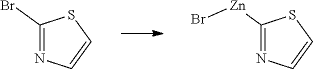

Under inert atmosphere, a reactor containing THF (200 mL) was charged with zinc (21.9 g, 335 mmol, 1,1 equiv.) and the addition port rinsed with additional THF (50 mL). With vigorous stirring at 23° C. (internal temperature), TMSCl (1.7 g, 15.2 mmol, 0.05 equiv.) was added slowly over approximiately 25 minutes, and the addition line rinsed with THF (10 mL). Vigorous stirring was continued for 30 minutes and then 2-bromothiazole (50 g, 304.8 mmol, 1.0 equiv.) was added over 2 h, and the addition line rinsed with THF (10 mL). Stirring was continued and the reaction was monitored by GC analysis for complete consumption of the 2-bromothiazole starting material. If necessary, the reaction was heated to reflux in order to complete conversion. The solution of bromo(thiazol-2-yl)zinc in THF can be filtered at ambient temperature under inert atmosphere to remove residual zinc, or used directly without filtration. The volume was adjusted by addition of THF to achieve a final volume of 305 mL, giving a 1M stock solution that is stable at room temperature when stored under inert atmosphere.

### Example 8

### Preparation of ethyl (4S)-4-(3-fluoro-2-methyl-phenyl)-6-methyl-2-thiazol-2-yl-1,4-dihydropyrimidine-5-carboxylate hydrobromide (Example 8)

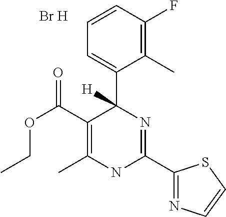

The title compound was prepared according to following scheme:

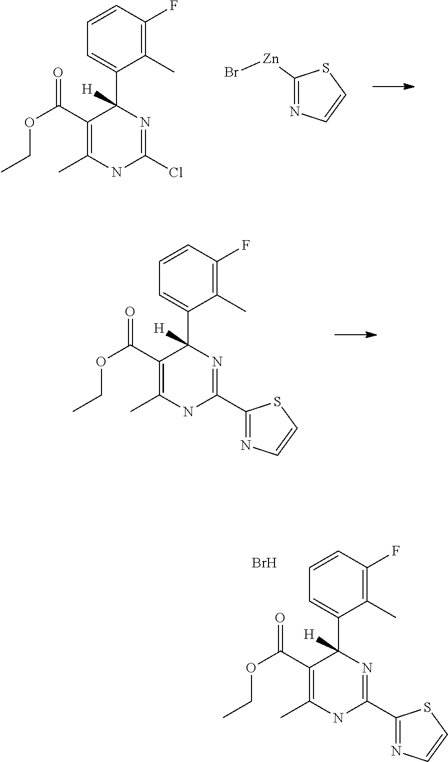

A reactor under inert atmosphere was charged with a solution of ethyl (4S)-2-chloro-4-(3-fluoro-2-methyl-phenyl)-6-methyl-1,4-dihydropyrimidine-5-carboxylate (21.26 g, 68.41 mmol, 1.0 equiv.) in toluene (0.36 M solution, 200 mL total volume), and then a portion bromo(thiazol-2-yl)zinc 1M solution in THF (6.8 mL, 0.1 equiv.), and then the catalyst dichloro[9,9-dimethyl-4,5-bis(diphenylphosphino)xanthene]palladium(II) (1.03 g, 1.4 mmol, 0.02 equiv.) was added as a solid, rinsing the addition port port with THF (8.9 g). The obtained red solution was heated to 70° C. (internal temperature). The remainder of bromo(thiazol-2-yl)zinc 1M solution in THF (130 mL, 1.9 equiv.) was added via infusion pump over 2 h, and the addition line rinsed with THF (8.9 g). The reaction was stirred for an addition 1 h, at which time the reaction was typically complete. The reaction promptly worked up by cooled to 23° C. (jacket temperature) and then washed with aqueous citric acid solution (13.14 g citric acid dissolved in 100 g water), followed two washes with water (200 mL total). The organic solution was partially concentrated under reduced pressure to a volume of 60 mL and then acetonitrile (157.2 g) was added and the reaction mixture once again concentrated to 60 mL. Acetonitrile (125.8 g) was added the resulting mixture was polish filtered. The filtered acetonitrile solution was warmed to 65° C. and then aqueous HBr (11.53 g of 48% w/w solution in water, 68.4 mmol, 1.0 equiv.) was added. Water was removed by distillation under reduced pressure (75-85° C. jacket temperature), with solvent replacement with acetonitrile. The reaction was concentrated to a minimal volume (approx. 40 mL) and then toluene (100 mL) added over 20 minutes (jacket temperature 85° C.). The resulting slurry was stirred for 1 h then cooled to 0° C. over 3 h, stirred for 1 h and the off-white to brown solid was isolated by filtration. The solid was washed with three portions of 5:1 toluene:acetonitrile (40 mL total volume), then dried at 50° C. under reduced pressure to provide 18.78 g (67.7% yield over two steps) of the title compound. (note: yield corrected for 92% assay of Ethyl (4S)-4-(3-fluoro-2-methyl-phenyl)-6-methyl-2-thioxo-3,4-dihydro-1H-pyrimidine-5-carboxylate starting material). 1H NMR (600 MHz, DMSO-d6) δ ppm 10.18-12.25 (m, 1H), 8.23 (m, 1H), 8.18 (m, 1H), 7.23-7.29 (m, 1H), 7.18-7.22 (m, 1H),7.08-7.15 (m, 1H), 5.91 (m, 1H), 3.85-4.05 (m, 2H), 2.49 (m, 3H), 2.43 (d, J=1.7 Hz, 3H), 1.04 (t, J=7.1 Hz, 3H); HRMS calcd C18 H18 F N3 O2 S [M+H]+: 360.1177, Found: 360.1181

### Example 9

### Preparation of 3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1 H-imidazo[1,5-a]pyrazin-2-yl]-2,2-dimethyl-propanoic acid (Example 9)

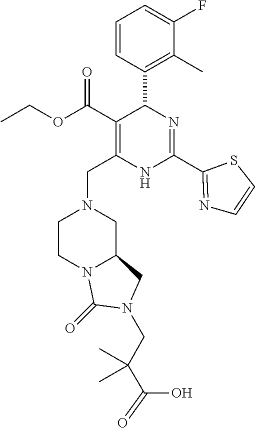

The title compound was prepared according to following scheme:

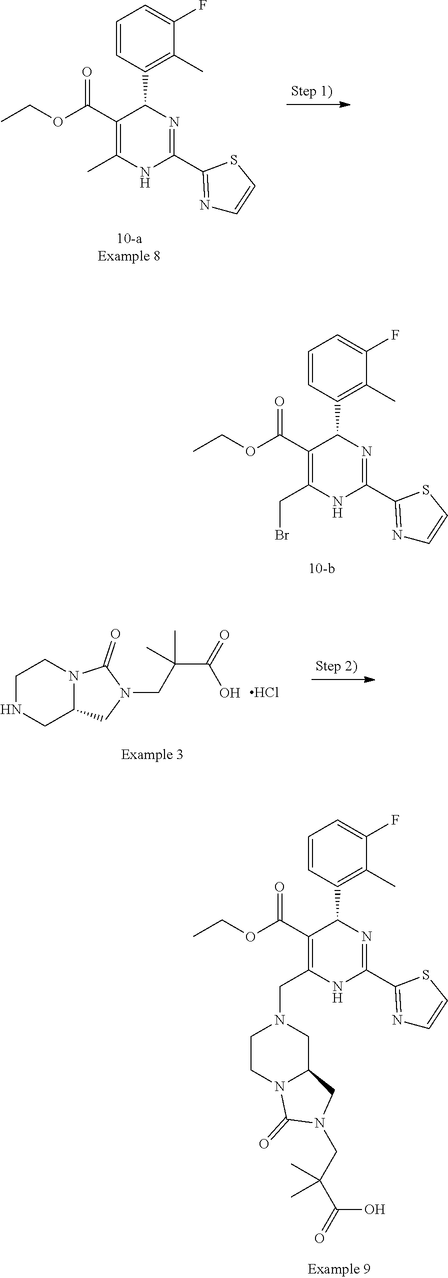 [SMILES: CCOC(=O)C1=C(C)NC(c2nccs2)=N[C@H]1c1cccc(F)c1C ; CCOC(=O)C1=C(CBr)NC(c2nccs2)=N[C@H]1c1cccc(F)c1C ; CC(C)(CN1C[C@@H]2CNCCN2C1=O)C(=O)O ; Cl ; CCOC(=O)C1=C(CN2CCN3C(=O)N(CC(C)(C)C(=O)O)C[C@@H]3C2)NC(c2nccs2)=N[C@H]1c1cccc(F)c1C]

### Example 9

### Step 1) Preparation of ethyl (4S)-6-(bromomethyl)-4-(3-fluoro-2-methyl-phenyl)-2-thiazol-2-yl-1,4-dihydropyrimidine-5-carboxylate (compound 10-b)

A 10 L flask equipped with mechanical stirrer, thermometer and nitrogen bubbler was charged with a solution of ethyl (4S)-4-(3-fluoro-2-methyl-phenyl)-6-methyl-2-thiazol-2-yl-1,4-dihydropyrimidine-5-carboxylate (706 mmol, compound 10-a) in DCM (4.0 L) from step 1). To the reaction mixture, heated to 32° C.-37° C, NBS (125.6 g, 706 mmol) was added in portions while maintaining the temperature at 35° C.-40° C. After 0.5 hour, additional batch of NBS (12.6 g, 70.6 mmol) was added to reaction mixture which was carefully monitored by HPLC until the conversion >95%. The resulting solution of compound 10-b was cooled to 10-20° C. and used directly for the next step. MS m/e=436.1/438.0 [M+H]⁺.

### Step 2) Preparation of 3-[(8a5)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid (Example 9)

A 10 L flask equipped with mechanical stirrer, thermometer and nitrogen bubbler was charged a solution of ethyl (4S)-6-(bromomethyl)-4-(3-fluoro-2-methyl-phenyl)-2-thiazol-2-yl-1,4-dihydropyrimidine-5-carboxylate in DCM from the last step. To the reaction mixture, cooled to 10-20° C., was added 3-[(8aS)-3-oxo-1,5,6,7,8,8a-hexahydroimidazo[1,5-a]pyrazin-2-yl]-2,2-dimethyl-propanoic acid hydrochloride (193 g, 635 mmol, purity: 91.6 wt %, Example 3) and followed by addition of triethanolamine (329 g, 2.33 mol) in DCM (350 mL) in portions below 25° C. The reaction mixture was stirred at 20° C.-30° C. for 16 hours. Then to the resulting reaction mixture was added water (1.25 L) and aqueous layer was adjusted to pH=3-4 using H₃PO₄ (85 wt %). After phase separation, the organic phase was washed with acidic water (1.25 L, H₃PO₄ solution with pH=2-3). After phase separation, the organic phase was extracted with aqueous H₃PO₄ solution (35 wt %, 1980 g) once and aqueous H₃PO₄ solution (35 wt %, 990 g) once. The combined aqueous layer was extracted with DCM (500 mL). To the aqueous layer, cooled to 0° C.-10° C., was added DCM (2.0 L). Then the aqueous layer was adjusted to pH=3-4 with aqueous NaOH solution (50 wt %, 770 g). After phase separation, the organic phase was washed with water (1.5 L) and filtered through celite (25 g) and then concentrated to about 500 mL in vacuo. The residue was diluted with ethanol (500 mL) and concentrated to about 500 mL in vacuo and this process was repeated one more time. Then the residue was diluted again with ethanol (1700 mL) and heated to 70-80 ° C. till all solid was dissolved. Water (2.20 L) was added to previous solution via an addition funnel while maintaining inner temperature between 60° C. and 78° C. Then the reaction mixture was cooled to 55° C. over 2 hours and maintained at 50° C. -55° C. for 1 hour, then cooled to 25° C. over 3 hours and stirred at 25° C. for another hour. The solid was collected by filtration and washed with ethanol/water (v/v=1/1, 250 g). The wet cake was dried in a vacuum oven (45° C.-55° C./Ca. 0.1Mpa with a nitrogen bleed) for 35 hours to afford the product Example 9 (260.0 g , purity: 99.1%, chiral purity: 99.8%, yield: 61.5%) as a light-yellow solid. ¹H NMR (400 MHz, DMSO-d⁶) δ 12.35 (s, 1H), 9.60 (s, 1H), 8.01 (d, J=3.2 Hz, 2H), 7.93 (d, J=3.2 Hz, 2H), 7.15-7.19 (m, 1H), 7.01-7.05 (m, 2H), 5.89 (s, 1H),3.87-4.00 (m, 4H), 3.62-3.73(m, 2H), 3.33-3.39 (m, 1H), 3.27 (d, J=14.0Hz, 1H), 3.16 (d, J=14.0Hz, 1H), 2.93-3.00 (m, 2H), 2.77-2.82 (m, 2H), 2.45 (t, J=1.6 Hz, 3H), 2.15 (d, J=11.2 Hz, 1H), 2.02 (d, J=11.2Hz, 1H), 1.03-1.08 (m, 9H); MS m/e =599.6 [M+H] ⁺.

### Example 10

### The H₃PO₄ Concentration and Equivalent Screening in the Acid-Base Work-Up of Step l)

The amount of H₃PO₄ in the acid-base work-up of step l) is essential and carefully designed to get the maximum recovery of API and minimum impurities. The concentration and equivalent of H₃PO₄ in step 2) of Example 9 were screened according to Table 1. The major impurity was Impurity 2 shown below.

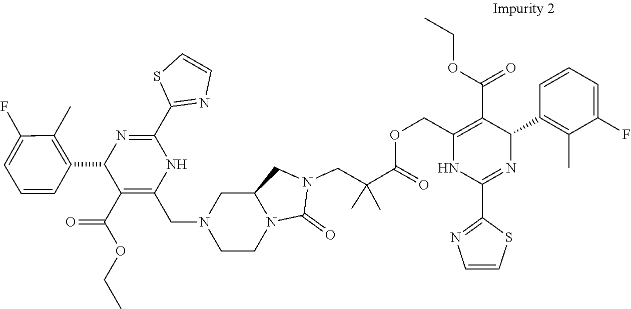

After the initial H₃PO₄ solution wash (pH=3-4 and pH=2-3), the purity in organic layer was Product/Impurity 2(Rt_{(impurity)}=19.4min)=71.9/1.38 (peak area %), the selected examples of further extractions with various H₃PO₄ concentration and equivalent were tested and shown in Table 1.

TABLE 1
H₃PO₄ concentration and equivalent screening

| | Aqueous layer purity | Organic layer purity |
|---|---|---|
| Concentration and | (peak area %) | (peak area %) |
| equivalent of H₃PO₄ | Product/Impurity 2 | Product/Impurity 2 |
| 30 wt % H₃PO₄ | 95.2/0.0 | 14.0/4.6 |
| 20 eq. | | |
| 35 wt % H₃PO₄ | 92.6/0.0 | 10.8/4.7 |
| 10 eq. | | |
| 35 wt % H₃PO₄ | 93.7/0.1 | 5.4/5.0 |
| 15 eq. | | |
| 35 wt % H₃PO₄ | 93.9/0.1 | 4.0/5.0 |
| 20 eq. | | |
| 40 wt % H₃PO₄ | 92.3/0.5 | 3.9/3.9 |
| 20 eq. | | |
| 45 wt % H₃PO₄ | 90.7/1.3 | 4.9/1.3 |
| 20 eq. | | |

The above study was tested with following HPLC parameters shown in Table 2.

TABLE 2
HPLC parameters

| Instrument | Agilent 1260 HPLC system with DAD detector | |
|---|---|---|
| Column | Waters Xbridge C8 (4.6 × 150 mm × 3.5 μm) | |
| Oven temperature | 30° | C. |

| Mobile phase | A: 0.12% TFA in water | | |
|---|---|---|---|
| | B: 0.12% TFA in ACN | | |
| | Time (min) | A % | B % |
| Gradient program | 0.00 | 80 | 20 |
| | 15.00 | 50 | 50 |
| | 20.00 | 10 | 90 |
| | 25.00 | 10 | 90 |
| | 25.01 | 80 | 20 |
| | 30.00 | 80 | 20 |

| Flow rate | 1.0 | mL/min |
|---|---|---|
| Detector | UV 299 nm | |
| Nominal concentration | 0.5 | mg/mL |
| Diluent | ACN:water = 1:1 | |
| Injection volume | 10 | μL |
| Run time | 30 | min |

According to the results shown in Table 1, the amount of H₃PO₄ in the acid-base work-up of step m) is directly related to the recovery of API and amount of impurities. Therefore, the particular concentration of H₃PO₄ was 35 wt % to 40 wt % and 10-15 equivalent of compound of formula (XVIII).

1. Process for the preparation of a compound of the formula (I), 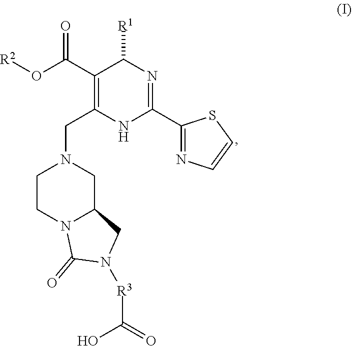
wherein
R¹ is phenyl, which is unsubstituted or substituted with one, two or three substituents independently selected from halogen and C₁₋₆alkyl;
R² is C₁₋₆alkyl;
R³ is —CₓH₂ₓ—;
x is 1, 2, 3, 4, 5, 6 or 7;
or pharmaceutically acceptable salt or diastereomer thereof;
comprising one or more of the following steps:
step a) the formation of compound (III), 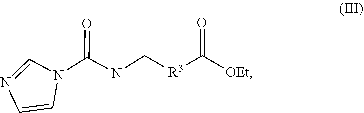
wherein R³ is —CₓH₂ₓ—; x is 1, 2, 3, 4, 5, 6 or 7;
step b) the formation of urea (V) 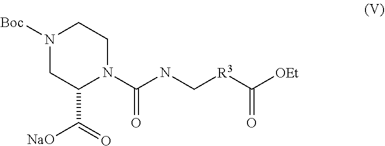
via the addition reaction of compound (III) and compound (IV) 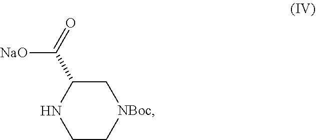
wherein R³ is —CₓH₂ₓ—; x is 1, 2, 3, 4, 5, 6 or 7;
step c) the formation of the hydantoin of formula (VI) via the cyclization reaction of urea (V), 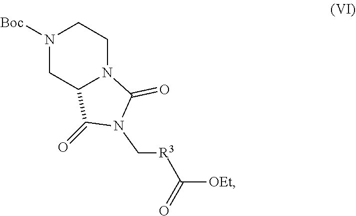
wherein R³ is —CₓH₂ₓ—; x is 1, 2, 3, 4, 5, 6 or 7;
step d) the formation of the urea of formula (VIII) via selective reduction of the compound of formula 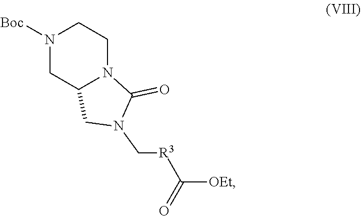
wherein R³ is —CₓH₂ₓ—; x is 1, 2, 3, 4, 5, 6 or 7; R is C₁₋₆alkyl;
steps e) and f) the formation of the compound of formula (IX) via hydrolysis of the compound of formula (VIII), 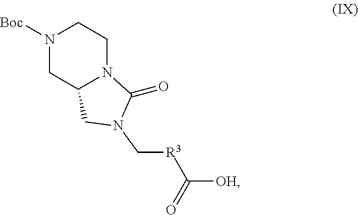
wherein R³ is —CₓH₂ₓ—; x is 1, 2, 3, 4, 5, 6 or 7; R is C₁₋₆alkyl;
step g) the formation of compound of formula (X) by de-protection of the compound of formula (IX), 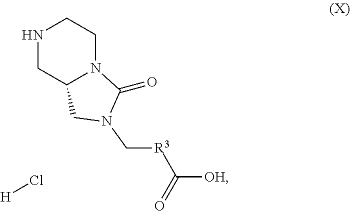
wherein R³ is —CₓH₂ₓ—; x is 1, 2, 3, 4, 5, 6 or 7;
step h) the formation of compound of formula (XIV) via the reaction of compounds (XI), (XII) and (XIII) in the presence of acid (XV), 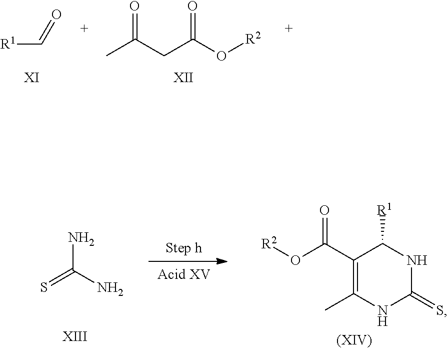
wherein R¹ is phenyl, which is unsubstituted or substituted with one, two or three substituents independently selected from halogen and C₁₋₆alkyl; R² is C₁₋₆alkyl;
step i) the formation of compound of formula (XVI), 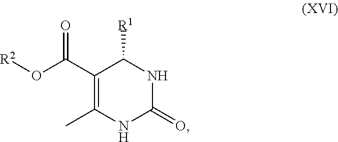
wherein R¹ is phenyl, which is unsubstituted or substituted with one, two or three substituents independently selected from halogen and C₁₋₆alkyl; R² is C₁₋₆alkyl;
step j) the formation of compound of formula (XVII), 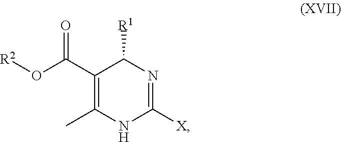
wherein R¹ is phenyl, which is unsubstituted or substituted with one, two or three substituents independently selected from halogen and C₁₋₆alkyl; R² is C₁₋₆alkyl; X is halogen, preferably chlorine;
step k) the formation of compound of formula (XVIII), 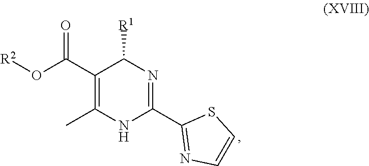
wherein R¹ is phenyl, which is unsubstituted or substituted with one, two or three substituents independently selected from halogen and C₁₋₆alkyl; R² is C₁₋₆alkyl;
step l) the formation of compound of formula (XIX) via the bromination reaction of compound of formula (XVIII), 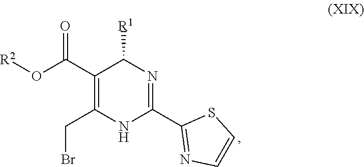
wherein R¹ is phenyl, which is unsubstituted or substituted with one, two or three substituents independently selected from halogen and C₁₋₆alkyl; R² is C₁₋₆alkyl;
step m) the formation of compound of formula (I) via the substitution reaction of compound of formula (XIX) with compound of formula (X), 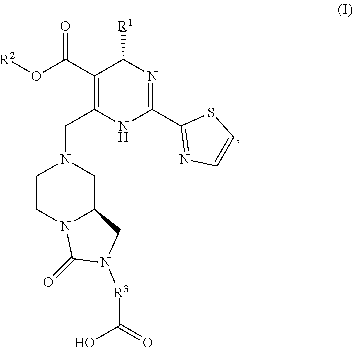
wherein R¹ is phenyl, which is unsubstituted or substituted with one, two or three substituents independently selected from halogen and C₁₋₆alkyl; R² is C₁₋₆alkyl; R³ is —CₓH₂ₓ—; x is 1, 2, 3, 4, 5, 6 or 7.

2. A process according to claim 1, wherein R¹ is chlorofluorophenyl, methylchlorophenyl or fluoromethylphenyl; R² is methyl or ethyl; R³ is dimethylethyl; or pharmaceutically acceptable salt or diastereomer thereof.

3. A process according to claim 1 or 2 for the synthesis of 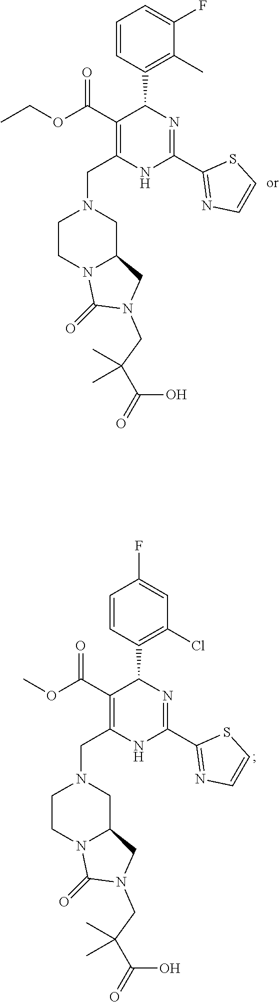 [SMILES: CCOC(=O)C1=C(CN2CCN3C(=O)N(CC(C)(C)C(=O)O)C[C@@H]3C2)NC(c2nccs2)=N[C@H]1c1cccc(F)c1C ; COC(=O)C1=C(CN2CCN3C(=O)N(CC(C)(C)C(=O)O)C[C@@H]3C2)NC(c2nccs2)=N[C@H]1c1ccc(F)cc1Cl]
or pharmaceutically acceptable salt or diastereomer thereof.

4. Process for the preparation of a compound of the formula (X), 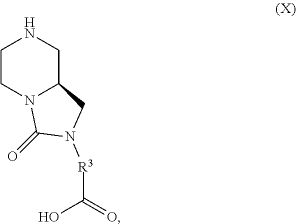
wherein
R³ is —CₓH₂ₓ—;
x is 1, 2, 3, 4, 5, 6 or 7;
or pharmaceutically acceptable salt, enantiomer or diastereomer thereof;
comprising one or more of the following steps:
step a) the formation of compound (III), 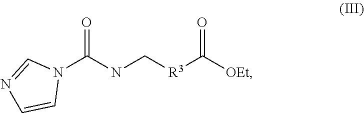
wherein R³ is —CₓH₂ₓ—; x is 1, 2, 3, 4, 5, 6 or 7;
step b) the formation of urea (V) 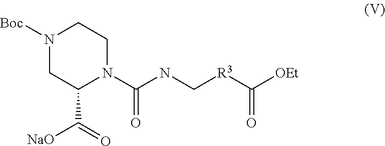
via the addition reaction of compound (III) and compound (IV) 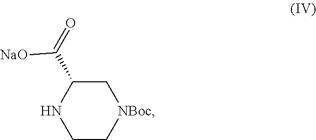
wherein R³ is —CₓH₂ₓ—; x is 1, 2, 3, 4, 5, 6 or 7;
step c) the formation of the hydantoin of formula (VI) via the cyclization reaction of urea (V), 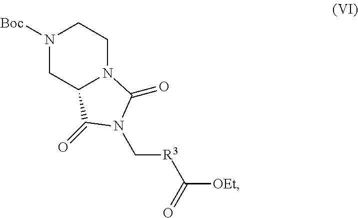
wherein R³ is —CₓH₂ₓ—; x is 1, 2, 3, 4, 5, 6 or 7;
step d) the formation of the urea of formula (VIII) via selective reduction of the compound of formula (VI), 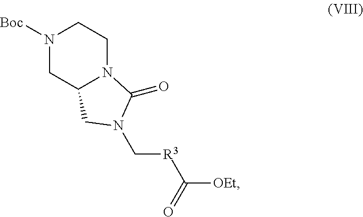
wherein R³ is —CₓH₂ₓ—; x is 1, 2, 3, 4, 5, 6 or 7; R is C₁₋₆alkyl;
steps e) and f) the formation of the compound of formula (IX) via hydrolysis of the compound of formula (VIII), 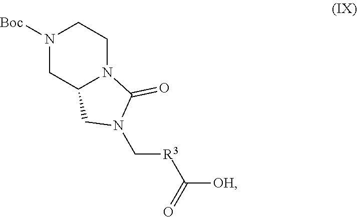
wherein R³ is —CₓH₂ₓ—; x is 1, 2, 3, 4, 5, 6 or 7; R is C₁₋₆alkyl;
step g) the formation of compound of formula (X) by de-protection of the compound of formula (IX), 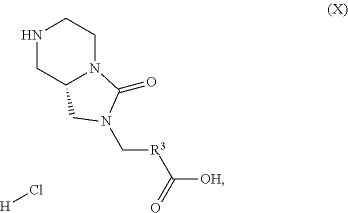
wherein R³ is —CₓH₂ₓ—; x is 1, 2, 3, 4, 5, 6 or 7.

5. A process according to claim 4, wherein R³ is dimethylethyl.

6. A process according to claim 4, wherein compound (X) is in the form of a pharmaceutically acceptable salt or diastereomer thereof.

7. A process according to any one of claims 1 to 6, characterized in that the formation of compound (III) in step a) is performed in the presence of a base in a solvent with a reagent, wherein the solvent is selected from 2-MeTHF, THF, IPAc, EA, DCM, DMF, toluene and anisole.

8. A process according to claim 7, wherein the base is selected from Na₂CO₃, NaOtPent, NaHCO₃, K₂CO₃, Na₃PO₄, K₃PO₄ and triethylamine (TEA).

9. A process according to claim 7 or 8, wherein the reagent is selected from CDI, phosgene, diphosgene, disuccinimidyl carbonate, and triphosgene.

10. A process according to any one of claims 1 to 9, characterized in that the formation of the hydantoin of formula (VI) in step c) is performed in the presence of an acid in an organic solvent, wherein the solvent is selected from 2-MeTHF, IPAc, EA, toluene, DCM, anisole, and DMF.

11. A process according to claim 10, wherein the acid is selected from boron trifluoride etherate, phosphoric acid, sulphuric acid, chlorosulphonic acid, trifluoroacetic acid, HBr, HCl, AlCl₃, TiCl₄, SnCl₄, ZrCl₄, TMSOTf, pivaloyl chloride, isobutyl chloroformate and oxalyl chloride.

12. A process according to any one of claims 1 to 11, characterized in that the formation of the urea of formula (VIII) in step d) is performed in the presence of a catalytic Lewis acid and a reducing agent, wherein the catalytic Lewis acid is selected InCl₃, YCl₃, ZnCl₂, Zn(OAc)₂, TMSCl, TiCl₄, ZrCl₄, AlCl₃, BF₃.THF, and BF₃.Et₂O.

13. A process according to claim 12, wherein the reducing agent is selected from lithium aluminum hydride, sodium dihydro-bis-(2-methoxyethoxy)aluminate, borane dimethylsulfide, phenylsilane, borane, borane dimethylsulphide complex and borane tetrahydrofurane complex.

14. A process according to any one of claims 1 to 13, characterized in that the compound of formula (IX) is synthesized in the presence of a solvent is selected from THF, MeTHF, TBME, toluene, anisole, isopropanol, methanol and ethanol and their mixtures with water.

15. A process according to any one of claims 1 to 14, characterized in that the formation of the compound of formula (X) in step g) is performed in the presence of HCl in a solvent.

16. A process according to claim 15, wherein the solvent is selected from DCM, toluene, dioxane, EtOAc, IPAc, IPA, 1-propanol, acetone, MIBK and mixed solvent of MIBK and acetone.

17. A process according to any one of claims 1 to 16, characterized in that the acid of formula (XV) in step h) is selected from the group consisting of (R)-3,3′-Bis(2,4,6-triisopropylphenyl)-1,1-binaphthyl-2,2′-diyl hydrogenphosphate, (S)-3,3′-Bis(2,4,6-triisopropylphenyl)-1,1-binaphthyl-2,2′-diylhydrogenphosphate, (R)-(−)-3,3′-Bis(triphenylsilyl)-1,1′-binaphthyl-2,2′-diyl hydrogenphosphate, (R)-(−)-VAPOL hydrogenphosphate, (+)-CSA, and (S)-(+)-1,1′-Binaphthyl-2,2′-diyl hydrogen phosphate, (R)-(−)-1,1′-Binaphthyl-2,2′-diyl hydrogen phosphate.

18. A process according to claim 17, characterized in that the acid of formula (XV) in step h) is (R)-(−)-3,3′-Bis(triphenylsilyl)-1,1′-binaphthyl-2,2′-diylhydrogenphosphate.
